(19) United States
(12) Patent Application Publication
(10) Pub. No.: US 2021/0382045 A1
(43) Pub. Date: Dec. 9, 2021

(54) DYNAMIC EXCITATION AND MEASUREMENT OF BIOCHEMICAL INTERACTIONS

(71) Applicant: Cardea Bio, Inc., San Diego, CA (US)

(72) Inventors: Kiana Aran, Pasadena, CA (US); Brett Goldsmith, San Diego, CA (US); Alexander Kane, Santa Cruz, CA (US); Regis Peytavi, Costa Mesa, CA (US)

(21) Appl. No.: 17/342,284

(22) Filed: Jun. 8, 2021

Related U.S. Application Data
(60) Provisional application No. 63/036,772, filed on Jun. 9, 2020.

(51) Int. Cl. G01N 33/543; G01N 21/77; G01N 21/45; G01N 27/414

(52) U.S. Cl. CPC G01N 33/54373; G01N 27/4145; G01N 21/45; G01N 21/7743

(57) ABSTRACT

Apparatuses, systems, and methods are disclosed for excitation and measurement of biochemical interactions. Excitation circuitry is configured to apply one or more excitation conditions to a biologically gated transistor that includes a channel, so that one or more output signals from the biologically gated transistor are affected by the excitation condition(s) and by a biochemical interaction of moieties within a sample fluid in contact with the channel surface. Measurement circuitry is configured to obtain information corresponding to the biochemical interaction occurring at one or more measurement distances greater than an electrostatic screening distance from the surface of the channel, by performing a plurality of time-dependent measurements of affected output signals, using a measurement bandwidth that corresponds to the measurement distances. An analysis module is configured to characterize one or more parameters of the biochemical interaction based on the time-dependent measurements.

### CROSS-REFERENCE TO RELATED APPLICATIONS

This application claims priority to U.S. Provisional Patent Application No. 63/036,772 titled “Dynamic Excitation And Measurement Of Biochemical Interactions” filed Jun. 9, 2020, U.S., which is hereby incorporated by reference to the extent legally allowable.

### FIELD

The subject matter disclosed herein relates to integrated electrical measurement systems and more particularly relates to dynamic excitation and measurement of biochemical interactions.

### BACKGROUND

Transistors and integrated circuits are rarely designed to work within liquid environments, and those that are typically work at very slow speeds. Typically, semiconductors coupled to a liquid environment wait for chemical equilibrium or are performed at a particular single frequency or with a very narrow bandwidth designed to characterize simple chemical interactions. Complex chemical and biochemical systems such as such as nucleic acids, proteins, and other compounds as well as biomolecular interactions contain multiple overlapping and dynamic timescales. Existing methods to characterize these systems include for example colorimetric assays that measure the color change of a reagent at the end point equilibrium of a bulk liquid phase reaction. Other methods may track the kinetics of a binding interaction optically by using specialized and expensive equipment to optically excite and measure the system. An integrated electronic equivalent is not yet available.

### BRIEF SUMMARY

Apparatuses are disclosed for excitation and measurement of biochemical interactions. In one or more examples, excitation circuitry is configured to apply one or more excitation conditions to a biologically gated transistor that includes a channel. One or more output signals from the biologically gated transistor may be affected by the one or more excitation conditions and by a biochemical interaction of moieties within a sample fluid in contact with a surface of the channel. In one or more further examples, measurement circuitry is configured to obtain information corresponding to the biochemical interaction occurring at one or more measurement distances including at least one measurement distance greater than an electrostatic screening distance from the surface of the channel, by performing a plurality of time-dependent measurements of at least one of the one or more output signals affected by the excitation conditions and the biochemical interaction, using a predetermined measurement bandwidth corresponding to the one or more measurement distances. In some examples, an analysis module is configured to characterize one or more parameters of the biochemical interaction based on the plurality of time-dependent measurements.

Systems are disclosed for excitation and measurement of biochemical interactions. In various examples, a biologically gated transistor includes a channel configured so that one or more output signals of the biologically gated transistor are affected by a biochemical interaction within a sample fluid, in response to application of the sample fluid in contact with a surface of the channel and application of one or more excitation conditions to the biologically gated transistor. In some examples, excitation circuitry is configured to apply one or more excitation conditions to the biologically gated transistor. In certain examples, measurement circuitry is configured to obtain information corresponding to the biochemical interaction occurring at one or more measurement distances including at least one measurement distance greater than an electrostatic screening distance from the surface of the channel, by performing a plurality of time-dependent measurements of at least one of the one or more output signals affected by the biochemical interaction, using a predetermined measurement bandwidth corresponding to the one or more measurement distances. In some examples, communication circuitry is configured to transmit information based on the plurality of time-dependent measurements to a remote data repository.

Methods are disclosed for excitation and measurement of biochemical interactions. A method, in one or more examples, includes providing a biologically gated transistor comprising a channel. In various examples, a method includes applying a sample fluid to the biologically gated transistor in contact with a surface of the channel. In some examples, a method includes applying one or more excitation conditions to the biologically gated transistor so that one or more output signals of the biologically gated transistor are affected by a biochemical interaction within the sample fluid. In some examples, a method includes obtaining information corresponding to the biochemical interaction by performing a plurality of time-dependent measurements of at least one of the one or more output signals affected by the biochemical interaction, using a predetermined measurement bandwidth corresponding to the one or more measurement distances. In certain examples, a method includes characterizing one or more parameters of the biochemical interaction based on the plurality of time-dependent measurements.

### BRIEF DESCRIPTION OF THE DRAWINGS

A more particular description of the examples briefly described above will be rendered by reference to specific examples that are illustrated in the appended drawings. Understanding that these drawings depict only some examples and are not therefore to be considered to be limiting of scope, the examples will be described and explained with additional specificity and detail through the use of the accompanying drawings, in which:

FIG. 1 is a schematic block diagram illustrating a system for excitation and measurement of biochemical interactions, in accordance with one or more examples of the present disclosure;

FIG. 2 is a schematic block diagram illustrating an apparatus for excitation and measurement of biochemical interactions, including a biologically gated transistor, in accordance with one or more examples of the present disclosure;

FIG. 3 is a schematic block diagram illustrating another apparatus for excitation and measurement of biochemical interactions, including another biologically gated transistor, in accordance with one or more examples of the present disclosure;

FIG. 4 is a schematic block diagram illustrating a further apparatus for excitation and measurement of biochemical interactions, including a further embodiment of biologically gated transistor;

FIG. 5 is a detail view of a region indicated in FIG. 4, illustrating a measurement distance and an electrostatic screening distance for measurement of biochemical interactions, in accordance with one or more examples of the present disclosure;

FIG. 6 is a schematic block diagram illustrating a measurement apparatus, in accordance with one or more examples of the present disclosure;

FIG. 7 is a schematic flow chart diagram illustrating a method for excitation and measurement of biochemical interactions, in accordance with one or more examples of the present disclosure;

FIG. 8 is a top view illustrating a first geometry for one or more liquid-gated graphene field effect transistors (“gFETs”), in accordance with one or more examples of the present disclosure;

FIG. 9 is a top view illustrating a second geometry for one or more liquid-gated gFETs, in accordance with one or more examples of the present disclosure;

FIG. 10 is a top view illustrating a third geometry for one or more liquid-gated gFETs, in accordance with one or more examples of the present disclosure;

FIG. 11 is a top view illustrating a fourth geometry for one or more liquid-gated gFETs, in accordance with one or more examples of the present disclosure;

FIG. 12 is a top view illustrating a fifth geometry for one or more liquid-gated gFETs, in accordance with one or more examples of the present disclosure;

FIG. 13 is a top view illustrating a sixth geometry for one or more liquid-gated gFETs, in accordance with one or more examples of the present disclosure;

FIG. 14 is a top view illustrating a seventh geometry for one or more liquid-gated gFETs, in accordance with one or more examples of the present disclosure;

FIG. 15 is a top view illustrating an eighth geometry for one or more liquid-gated gFETs, in accordance with one or more examples of the present disclosure;

FIG. 16 is a top view illustrating a ninth geometry for one or more liquid-gated gFETs;

FIG. 17 is a top view illustrating a tenth geometry for one or more liquid-gated gFETs, in accordance with one or more examples of the present disclosure;

FIG. 18 is a top view illustrating an eleventh geometry for one or more liquid-gated gFETs, in accordance with one or more examples of the present disclosure;

FIG. 19 is a top view illustrating a twelfth geometry for one or more liquid-gated gFETs, in accordance with one or more examples of the present disclosure;

FIG. 20 is a top view illustrating a thirteenth geometry for one or more liquid-gated gFETs, in accordance with one or more examples of the present disclosure;

FIG. 21 is a top view illustrating a fourteenth geometry for one or more liquid-gated gFETs, in accordance with one or more examples of the present disclosure;

FIG. 22 is a top view illustrating a fifteenth geometry for one or more liquid-gated gFETs, in accordance with one or more examples of the present disclosure;

FIG. 23 is a top view illustrating a sixteenth geometry for one or more liquid-gated gFETs, in accordance with one or more examples of the present disclosure;

FIG. 24 is a top view illustrating a seventeenth geometry for one or more liquid-gated gFETs, in accordance with one or more examples of the present disclosure;

FIG. 25 is a top view illustrating an eighteenth geometry for one or more liquid-gated gFETs, in accordance with one or more examples of the present disclosure;

FIG. 26 is a top view illustrating a nineteenth geometry for one or more liquid-gated gFETs, in accordance with one or more examples of the present disclosure;

FIG. 27 is a top view illustrating a twentieth geometry for one or more liquid-gated gFETs, in accordance with one or more examples of the present disclosure;

FIG. 28 is a top view illustrating a twenty-first geometry for one or more liquid-gated gFETs, in accordance with one or more examples of the present disclosure;

FIG. 29 is a top view illustrating a twenty-second geometry for one or more liquid-gated gFETs, in accordance with one or more examples of the present disclosure; and

FIG. 30 is a top view illustrating a twenty-third geometry for one or more liquid-gated gFETs, in accordance with one or more examples of the present disclosure.

### DETAILED DESCRIPTION

As will be appreciated by one skilled in the art, aspects of the disclosure may be implemented as a system, method, or program product. Accordingly, implementations may take the form of an entirely hardware implementation, an entirely software implementation (including firmware, resident software, micro-code, etc.) or an implementation combining software and hardware aspects that may all generally be referred to herein as a “circuit,” “module,” or “system.” Furthermore, example implementations may take the form of a program product implemented in one or more computer readable storage devices storing machine readable code, computer readable code, and/or program code, referred hereafter as code. The storage devices may be tangible, non-transitory, and/or non-transmission. The storage devices may not embody signals. In certain implementation, the storage devices only employ signals for accessing code.

Certain of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in code and/or software for execution by various types of processors. An identified module of code may, for instance, comprise one or more physical or logical blocks of executable code which may, for instance, be organized as an object, procedure, or function. Nevertheless, the executables of an identified module need not be physically located together, but may comprise disparate instructions stored in different locations which, when joined logically together, comprise the module and achieve the stated purpose for the module.

Indeed, a module of code may be a single instruction, or many instructions, and may even be distributed over several different code segments, among different programs, and across several memory devices. Similarly, operational data may be identified and illustrated herein within modules, and may be implemented in any suitable form and organized within any suitable type of data structure. The operational data may be collected as a single data set, or may be distributed over different locations including over different computer readable storage devices. Where a module or portions of a module are implemented in software, the software portions are stored on one or more computer readable storage devices.

Any combination of one or more computer readable medium may be utilized. The computer readable medium may be a computer readable storage medium. The computer readable storage medium may be a storage device storing the code. The storage device may be, for example, but not limited to, an electronic, magnetic, optical, electromagnetic, infrared, holographic, micromechanical, or semiconductor system, apparatus, or device, or any suitable combination of the foregoing.

More specific examples (a non-exhaustive list) of the storage device would include the following: an electrical connection having one or more wires, a portable computer diskette, a hard disk, a random-access memory (RAM), a read-only memory (ROM), an erasable programmable read-only memory (EPROM or Flash memory), a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Code for carrying out operations for various example implementations may be written in any combination of one or more programming languages including an object-oriented programming language such as Python, Ruby, Java, Smalltalk, C++, or the like, and conventional procedural programming languages, such as the “C” programming language, or the like, and/or machine languages such as assembly languages. The code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

A component, as used herein, comprises a tangible, physical, non-transitory device. For example, a component may be implemented as a hardware logic circuit comprising custom VLSI circuits, gate arrays, or other integrated circuits; off-the-shelf semiconductors such as logic chips, transistors, or other discrete devices; and/or other mechanical or electrical devices. A component may also be implemented in programmable hardware devices such as field programmable gate arrays, programmable array logic, programmable logic devices, or the like. A component may comprise one or more silicon integrated circuit devices (e.g., chips, die, die planes, packages) or other discrete electrical devices, in electrical communication with one or more other components through electrical lines of a printed circuit board (PCB) or the like. Each of the modules described herein, in certain examples, may alternatively be implemented as one or more components.

A circuit, or circuitry, as used herein, comprises a set of one or more electrical and/or electronic components providing one or more pathways for electrical current. In certain examples, circuitry may include a return pathway for electrical current, so that a circuit is a closed loop. In some examples, however, a set of components that does not include a return pathway for electrical current may be referred to as a circuit or as circuitry (e.g., an open loop). For example, an integrated circuit may be referred to as a circuit or as circuitry regardless of whether the integrated circuit is coupled to ground (as a return pathway for electrical current) or not. In various examples, circuitry may include an integrated circuit, a portion of an integrated circuit, a set of integrated circuits, a set of non-integrated electrical and/or electrical components with or without integrated circuit devices, or the like. In various examples, a circuit may include custom VLSI circuits, gate arrays, logic circuits, or other integrated circuits; off-the-shelf semiconductors such as logic chips, transistors, or other discrete devices; and/or other mechanical or electrical devices. A circuit may also be implemented as a synthesized circuit in a programmable hardware device such as field programmable gate array, programmable array logic, programmable logic device, or the like (e.g., as firmware, a netlist, or the like). A circuit may comprise one or more silicon integrated circuit devices (e.g., chips, die, die planes, packages) or other discrete electrical devices, in electrical communication with one or more other components through electrical lines of a printed circuit board (PCB) or the like. Each of the modules described herein, in certain examples, may be embodied by or implemented as a circuit.

Reference throughout this specification to “one example,” “an example,” “one implementation,” “an implementation” or similar language means that a particular feature, structure, or characteristic described in connection with the example or implementation is included in at least one example or implementation. Thus, appearances of the phrases “in one example,” “in an example,” and similar language throughout this specification may, but do not necessarily, all refer to the same example or implementation, but mean “one or more but not all implementations” unless expressly specified otherwise. The terms “including,” “comprising,” “having,” and variations thereof mean “including but not limited to,” unless expressly specified otherwise. An enumerated listing of items does not imply that any or all of the items are mutually exclusive, unless expressly specified otherwise. The terms “a,” “an,” and “the” also refer to “one or more” unless expressly specified otherwise.

Furthermore, the described features, structures, or characteristics of the examples or implementations may be combined in any suitable manner. In the following description, numerous specific details are provided, such as examples of programming, software modules, user selections, network transactions, database queries, database structures, hardware modules, hardware circuits, hardware chips, etc., to provide a thorough understanding of embodiments. One skilled in the relevant art will recognize, however, that implementation may be practiced without one or more of the specific details, or with other methods, components, materials, and so forth. In other instances, well-known structures, materials, or operations are not shown or described in detail to avoid obscuring aspects of an example implementation.

Aspects of the example implementations are described below with reference to schematic flowchart diagrams and/or schematic block diagrams of methods, apparatuses, systems, and program products according to examples. It will be understood that each block of the schematic flowchart diagrams and/or schematic block diagrams, and combinations of blocks in the schematic flowchart diagrams and/or schematic block diagrams, can be implemented by code. This code may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the schematic flowchart diagrams and/or schematic block diagrams block or blocks.

The code may also be stored in a storage device that can direct a computer, other programmable data processing apparatus, or other devices to function in a particular manner, such that the instructions stored in the storage device produce an article of manufacture including instructions which implement the function/act specified in the schematic flowchart diagrams and/or schematic block diagrams block or blocks.

The code may also be loaded onto a computer, other programmable data processing apparatus, or other devices to cause a series of operational steps to be performed on the computer, other programmable apparatus, or other devices to produce a computer implemented process such that the code which execute on the computer or other programmable apparatus provide processes for implementing the functions/acts specified in the flowchart and/or block diagram block or blocks.

The schematic flowchart diagrams and/or schematic block diagrams in the Figures illustrate the architecture, functionality, and operation of possible implementations of apparatuses, systems, methods, and program products according to various examples. In this regard, each block in the schematic flowchart diagrams and/or schematic block diagrams may represent a module, segment, or portion of code, which comprises one or more executable instructions of the code for implementing the specified logical function(s).

It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding examples. Indeed, some arrows or other connectors may be used to indicate only the logical flow of the depicted example. For instance, an arrow may indicate a waiting or monitoring period of unspecified duration between enumerated steps of the depicted example. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and code.

The description of elements in each figure may refer to elements of proceeding figures. Like numbers refer to like elements in all figures, including alternate examples of like elements.

As used herein, a list with a conjunction of “and/or” includes any single item in the list or a combination of items in the list. For example, a list of A, B, and/or C includes only A, only B, only C, a combination of A and B, a combination of B and C, a combination of A and C or a combination of A, B and C. As used herein, a list using the terminology “one or more of” includes any single item in the list or a combination of items in the list. For example, one or more of A, B and C includes only A, only B, only C, a combination of A and B, a combination of B and C, a combination of A and C or a combination of A, B and C. As used herein, a list using the terminology “one of” includes one and only one of any single item in the list. For example, “one of A, B and C” includes only A, only B or only C and excludes combinations of A, B and C. As used herein, “a member selected from the group consisting of A, B, and C,” includes one and only one of A, B, or C, and excludes combinations of A, B, and C.” As used herein, “a member selected from the group consisting of A, B, and C and combinations thereof” includes only A, only B, only C, a combination of A and B, a combination of B and C, a combination of A and C or a combination of A, B and C.

### Definitions

The term “biomolecule,” as used herein, refers to any molecule that is produced by a biological organism or which is synthetically produced to simulate, represent, or work along with molecules produced by biological organisms, including large polymeric molecules such as proteins, polysaccharides, lipids, and nucleic acids (DNA and RNA) as well as small molecules such as primary metabolites, secondary metabolites, and other natural products.

The term “moiety,” as used herein, refers to a part of a molecule. For example, a moiety may be an active part of a drug molecule, an inactive part of a drug molecule, a part of an enzyme molecule that binds to the enzyme's substrate, a part of the substrate molecule that binds to the enzyme, another part of an enzyme or substrate, a region of a DNA or RNA molecule, an antigen-binding region (Fab) of an antibody, a crystallizable region (Fc) of an antibody, or the like. In the plural form, the term “moieties” may be used to refer to multiple types of moieties (e.g., an enzyme moiety and a substrate moiety) or to the same type of moiety for multiple molecules (e.g., a moiety of a protein that is present in multiple types or versions of protein). Moieties may be referred to as “within” a fluid if the moieties are in contact with molecules of the fluid. For example, a moiety within a fluid may be dissolved or suspended within the fluid, or may be disposed on the surface of a solid, where the fluid is in contact with that surface so that the moiety on the surface can interact with other molecules within the fluid.

The term “biochemical interaction,” as used herein, refers to a chemical or physical interaction of one or more moieties of biomolecules. A biochemical interaction may include an interaction of moieties with other moieties (e.g., an enzyme linking to a substrate), or may include an interaction of moieties with an applied physical condition such as a temperature or electric field (e.g., movement of moieties in a protein in response to temperature).

The term “biologically gated transistor,” as used herein, refers to a transistor where current between source and drain terminals, through at least one channel, is capable of being gated, modulated, or affected by a presence of a biomolecule or a biochemical interaction of moieties within a sample fluid in contact with (which may include within measurement distance of) a surface of the channel. In other words, in various examples, the term “in contact with a surface of the channel” can refer both to a substance being in fluid contact with the surface of the channel as well as the substance being within measurement distance of the channel. For example, in certain examples, a surface of the channel may covered by a membrane, a gel, or even a solid state layer, and may, the sample fluid may be understood to be “in contact with the surface of the channel” if relevant analytes within the sample fluid are within measurement distance of the channel whether or not the sample fluid is in fluid contact with the surface of the channel, The term “biologically gated transistor” may be used to refer to such a device in use, with a sample fluid applied to the surface of the channel (or within measurement distance of the channel), or to the same device before the sample fluid has been applied.

The term “output signal,” as used herein, refers to a measurable or detectable electrical signal from a biologically gated transistor, or to a result that can be calculated based on the measurable or detectable signal. For example, an output signal may be a voltage at one or more terminals of a biologically gated transistor, a current at one or more biologically gated transistor, a capacitance, inductance, or resistance (calculated based on applied and measured voltages and currents), a complex-valued impedance, a complex impedance spectrum, an electrochemical impedance spectrum, a Dirac voltage, a power spectral density, one or more network parameters (such as S-parameters or h-parameters), or the like.

The term “excitation condition,” as used herein, refers to a physical, electrical, or chemical condition applied to a biologically gated transistor or to a sample for measurement by a biologically gated transistor. Excitation conditions may affect a biochemical interaction, which in turn may affect one or more output signals from the biologically gated transistor. For example, excitation conditions may include voltages, currents, frequencies, amplitudes, phases, or waveforms of electrical signals applied to a biologically gated transistor, one or more temperatures, one or more fluid flow rates, one or more wavelengths of electromagnetic radiation, or the like.

The term “distance,” as used herein with reference to a distance from the surface of a channel in a biologically gated transistor, refers to a distance between a point (e.g., in the sample fluid), and the closest point of the channel surface to that point. For example, the distance from the surface of the channel to a point directly above the channel in the sample fluid is the distance between a point on the channel surface to the point in the sample fluid along a line that is normal (perpendicular) to the channel surface.

The term “measurement distance,” as used herein, refers to a distance from the surface of a channel in a biologically gated transistor, such that at least some aspect or portion of a biochemical interaction occurring at the measurement distance affects an output signal in a way that is detectable by a measurement apparatus. In other words, output signals from a biologically gated transistor are sensitive to biochemical events occurring at or within the measurement distance from the surface of a channel. Whether an effect on an output signal is detectable by a measurement apparatus may depend on actual sensitivity of the measurement apparatus, on a noise level for noise in the output signal, the extent to which the output signal is affected by aspects or portion of a biochemical interaction occurring closer to the channel surface, or the like. Whether an effect on an output signal is detectable by a measurement apparatus may be based on a predetermined threshold for detection or sensitivity, which may be signal to noise ratio, a ratio between effects on the output signal caused by events at a distance from the channel to effects on the output signal caused by events at the channel surface, or the like. In some examples, a measurement distance may depend on excitation conditions, or may be frequency dependent.

The term “electrostatic screening distance” as used herein, refers to a measurement distance for a biologically gated transistor for steady state (e.g., constant voltage or direct current) or low-frequency (e.g., less than 10 Hz) excitation conditions and measurements. One or more layers of ions may form near the surface of a channel of a biologically gated transistor when a fluid is applied in contact with the channel surface. For example, a double layer of ions may include a first layer of ions attracted or adsorbed to the channel surface and a second layer of ions attracted to the ions in the first layer. Or, if the channel has been functionalized by immobilizing certain molecules or moieties (e.g., proteins, peptides, surfactants, polymers such as polyethylene glycol, or the like) to the channel surface, forming an ion-permeable layer with a net charge, then ions from the fluid may diffuse into the ion-permeable layer of immobilized molecules or moieties due to the Gibbs-Donnan effect, forming a Donnan equilibrium region, and creating a measurable Donnan capacitance. In either case, charges near the channel surface may act as a “screen” between the channel and the bulk of the sample fluid. Thus, steady-state, or low-frequency excitation and measurement may result in a measurement apparatus detecting effects on output signals for only the aspects or portions of a biochemical interaction that occur in or near the double layer, or the Donnan equilibrium region, and the electrostatic screening distance may be based on the thickness (e.g., Debye length) for a double layer and/or a Donnan equilibrium region.

The term “measurement bandwidth” as used herein refers to a band or range of frequencies for which output signals of a biologically gated transistor are measured. For example, where discrete samples of the output signals are measured at a sampling rate, the measurement bandwidth may be a range from 0 Hz to half the sampling rate.

The term “bias” as used herein refers to an electrical signal or waveform applied to an electrode or terminal of a biologically gated transistor, such as a source, drain, counter electrode, or another electrode. The term “programmable bias” is used to refer to a bias that is capable of being changed, varied, or modulated by the circuitry that applies the bias. Examples of programmable biases include a constant voltage or current selected by bias circuitry, a square wave, a sine wave, a more complicated waveform such as a sum of sine waves of various amplitudes, frequencies, and phases (possibly also including a zero-frequency or DC offset component), or the like.

The term “CMOS” as used herein, refers to complementary metal oxide semiconductor technology, devices, and/or processing steps, as well as to certain technologies, devices and/or processing steps separate from a CMOS process, which utilize processing tools usable in the CMOS processing steps. CMOS technology may be used to fabricate digital, analog, or mixed signal circuitry. Furthermore, the term “CMOS under <<other technology>>”, as in “CMOS under graphene”, indicates that certain circuitry using the “other technology” (e.g., graphene) and CMOS circuitry may be stacked one above the other. In some examples, a first portion of the other technology (e.g., graphene) circuitry and the CMOS circuitry may be stacked one above the other and a second portion of CMOS circuitry may be disposed at a horizontal distance from the other technology (e.g., graphene) circuitry.

Various methods for investigating and characterizing biomolecules or biomolecular interactions may be expensive or complex. For example, colorimetric assays or PCR-based assays may involve expensive or complex reagents, large testing devices, or the like. Tests based on optical spectroscopy may involve labeling of biological material to differentiate parts of the sample. Labeling may chemically change the sample, and may involve extensive sample purification and processing. The result may be a snapshot in time of the state of the sample, without information about how the biological or chemical aspects of the sample change over time. Optical techniques for real-time monitoring of biological or chemical changes may be difficult and expensive. For example, an optical biomolecule conformational analysis platform that uses femtosecond lasers to drive second harmonic generation on an array of dyes functionalized to a biomolecule may provide real-time information, but may require highly specialized optical equipment, and a deep mastery and understanding of the complete surface chemistry of the biomolecule.

Certain electronic or electrical biosensing methods may similarly provide limited information, or may involve great complexity and expertise. For example, electrical biosensing at a constant voltage or frequency may record one type of information at the expense of ignoring other available information that might be available using optical or mass spectroscopy. More sophisticated electronic biosensing has been carried out in single-molecule experiments performed in nanogaps or on carbon nanotubes, to probe the dynamics (such as activity and conformation) of biomolecules. These techniques for obtaining real-time or dynamic information have involved specialty lab equipment with very low throughput, and skills and knowledge associated with PhD level nanotechnologists.

By contrast, electronic measurement or characterization of biomolecular interactions using biologically gated transistors, as disclosed herein, may provide real-time information about biological and/or chemical dynamics, with low cost and complexity. Sensors including biologically gated transistors may be built using traditional electronics manufacturing techniques, leading to lower costs. Some tests may be label-free, reducing the need for complex or multi-step reactions that change the sample, and the need for certain reagents. Tools for label-free measurements may be capable of performing a wide variety of chemical and biochemical assays, leading to lower overall cost for individual measurements.

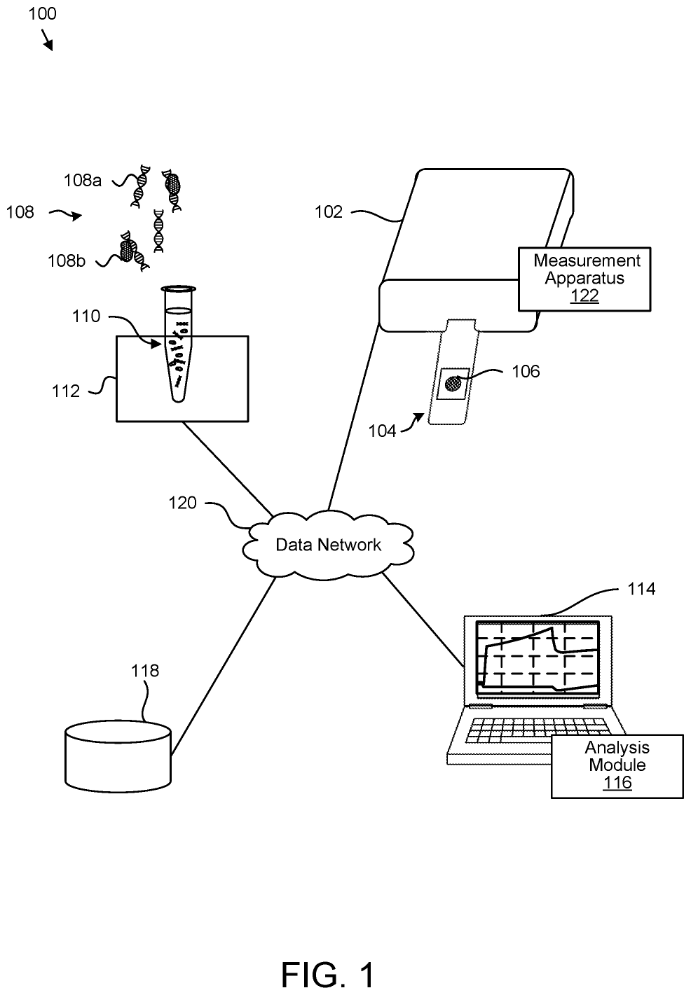

FIG. 1 is a schematic block diagram illustrating a system 100 for excitation and measurement of biochemical interactions, in accordance with one or more examples of the present disclosure. The system 100, in the depicted example, includes, one or more chip-based biosensors 104, a chip reader device 102, a sample prep apparatus 112, a computing device 114, a remote data repository 118, and a data network 120.

A chip-based biosensor 104, in the depicted example, includes one or more biologically gated transistors 106, which are described in further detail below. In various examples, a chip-based biosensor 104 is a device including one or more solid 2D two-dimensional sensor elements (such as biologically gated transistors 106 and/or other sensor elements) arranged on a solid support. The sensor elements may respond directly or indirectly to the presence of a proximate biochemical or biomolecular analyte or interaction, or both, in a sample on or sufficiently proximate to the sensor elements to produce an electrical or electromagnetic response signal suitable for amplification, filtering, digitization, and other analog and digital signal processing operations.

In some examples, a chip-based biosensor 104 may include a plurality of transistors and a plurality of detection moieties where at least one of the transistors is a biologically gated transistor 106. In certain examples, a chip-based biosensor 104 may include one or more additional sensors alongside biologically gated transistors 106. For example, various types of sensors may be included that use terahertz spectroscopy, surface-enhanced spectroscopy, quartz crystal microbalance, grating-coupled interferometry, and so forth. In some examples, a chip-based biosensor 104 may include further components such as a flow cell or fluid propulsion mechanism.

In the depicted example, the chip reader device 102 includes circuitry for communicating with (e.g., sending electrical signals to or receiving electrical signals from) components of the chip-based biosensor 104. For example, a chip-based biosensor 104 may include a chip or integrated circuit with one or more biologically gated transistors 106, mounted to a printed circuit board with electrical contacts at one edge. A socket in the chip reader device 102 may include matching contacts, so that the chip-based biosensor 104 can be plugged into or removed from the chip reader device 102. Various other or further types of connectors may be used to provide a detachable coupling between a chip-based biosensor 104 and a chip reader device 102.

In a further example, the chip reader device 102 may include circuitry for communicating via the data network 120. For example, the chip reader device 102 may communicate information about measurements performed using the chip-based biosensor 104 to the computing device 114 and/or to a remote data repository 118, over the data network. The data network 120, in various examples, may be the Internet, or may be another network such as a wide area network, metropolitan area network, local area network, virtual private network, or the like. In another example, the chip reader device 102 may communicate information in another way, in addition to or in place of communicating over a data network 120. For example, the chip reader device 102 may display or print information, save information to a removable data storage device, or the like.

In the depicted example, a measurement apparatus 122 is implemented by the chip-based biosensor 104 and/or the chip reader device 102. In various examples, a measurement apparatus 122 may include excitation circuitry to apply excitation conditions to a biologically gated transistor 106. Output signals from the biologically gated transistor 106 (such as electrical currents, voltages, capacitances, impedances, or the like) may be affected by the excitations and by a biochemical interaction within a sample fluid 110 applied to the biologically gated transistor 106. The measurement apparatus 122 may include measurement circuitry to obtain information about or corresponding to the biochemical interaction. The measurement circuitry may perform a plurality of time-dependent measurements of at least one of the output signals that are affected by the excitation conditions and the biochemical interaction.

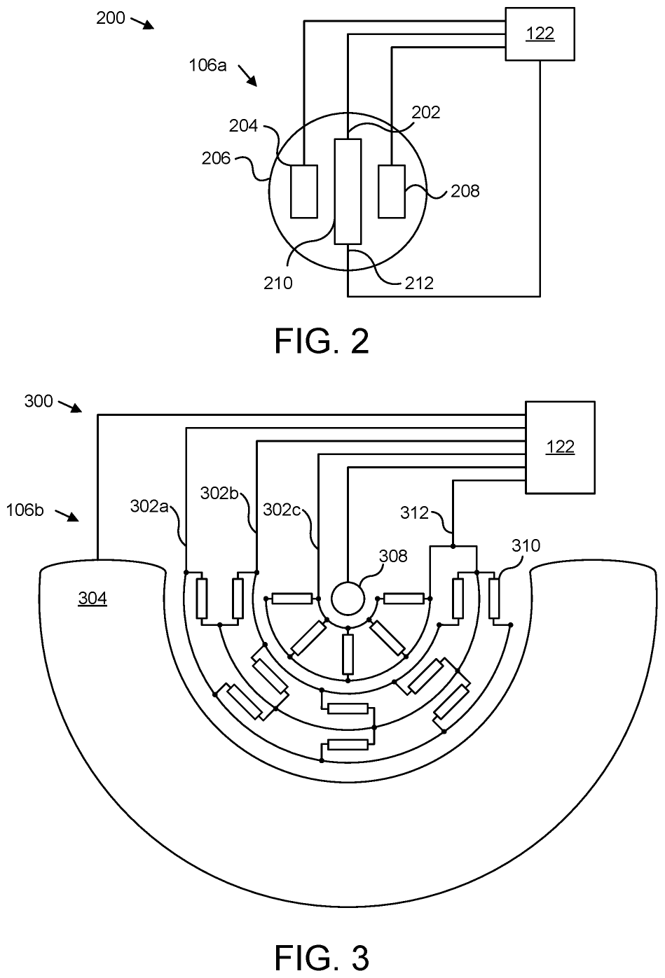

A measurement bandwidth may be based on a sample rate for performing the time-dependent measurements. For example, a measurement apparatus 122 may be capable of “seeing” (e.g., observing or detecting information about) real-time information about the biochemical interaction for aspects or characteristics of the interaction with frequencies in a measurement bandwidth between 0 Hz and a frequency of half the sample rate. In various examples, wide-bandwidth sampling (e.g., with a predetermined measurement bandwidth) may provide real-time information that cannot be obtained by making constant-voltage or single-frequency (narrowband) measurements. In some examples, the information thus obtained may be comparable to real-time information obtained by using optical spectroscopy or mass spectroscopy, but without the high cost and complexity associated with optical or mass spectroscopy. Various examples of a measurement apparatus 122 are described in further detail below with reference to FIGS. 2-7.

In some examples, a chip-based biosensor 104 may include the measurement apparatus 122. For example, excitation circuitry and/or measurement circuitry may be provided on the same chip as a biologically gated transistor 106, or on the same package, on the same printed circuit board, or the like, as part of a chip-based biosensor 104. In another example, the chip reader device 102 may include the measurement apparatus 122. For example, excitation circuitry and/or measurement circuitry may be provided in a chip reader device 102 so that the excitation circuitry and/or measurement circuitry is reusable with multiple chip-based biosensors 104.

In another example, a chip-based biosensor 104 and a chip reader device 102 may both include portions of a measurement apparatus 122. For example, the chip-based biosensor 104 may include portions of the excitation circuitry, such as a resistive heater for temperature control of the biologically gated transistor 106, and the chip reader device 102 may include other portions of the excitation circuitry such as a voltage or current source. In various examples, excitation circuitry, measurement circuitry and/or other components of a measurement apparatus 122 may be disposed between a chip-based biosensor 104 and a chip reader device 102 in various other or further ways.

Additionally, although the system 100 in the depicted example includes a chip-based biosensor 104 that may be coupled to or removed from a chip reader device 102, the functions and/or components of a chip-based biosensor 104 and a chip reader device 102 may be integrated into a single device in another example. Conversely, in some examples, a system may include multiple devices rather than a single chip reader device 102. For example, excitation circuitry and/or measurement circuitry for a measurement apparatus 122 may include lab bench hardware such as source measure units, function generators, bias tees, chemical impedance analyzers, lock-in amplifiers, data acquisition devices, or the like, which may be coupled to a chip-based biosensor 104.

The sample prep apparatus 112, in the depicted example, is configured to automatically or semi-automatically prepare the sample fluid 110. In some examples, a sample prep apparatus 112 may include automated dispensing equipment such as a dispensing robot and/or a fluidic system. In some examples, a sample prep apparatus 112 may include its own controller and user interface for setting sample prep parameters such as incubation time and temperature for the sample fluid 110. In some examples, a sample prep apparatus 112 may be controlled via the data network 120. For example, the computing device 114 or the measurement apparatus 122 may control the sample prep apparatus 112.

In another example, a system 100 may omit a sample prep apparatus 112, and a sample fluid 110 may be manually prepared. In some examples, preparing a sample fluid 110 may include obtaining or preparing a sample of a fluid in which a biochemical interaction may be observed (or the absence of a biochemical interaction may be detected). In some examples, a sample fluid 110 once obtained may be applied directly to the chip-based biosensor 104. For example, in some examples, the chip-based biosensor 104 may be used to characterize or measure a biochemical interaction in blood, and the blood may be applied to the chip-based biosensor 104 as the sample fluid 110. In another example, further sample prep steps to prepare a sample fluid 110 may include the addition of reagents, concentration or dilution, heating or cooling, centrifuging, or the like. Various other or further preparation techniques may be used to prepare a sample fluid 110 for use with a measurement apparatus 122.

The sample fluid 110, in various examples, may include one or more types of biomolecules 108. Biomolecules 108, in various examples, may be any molecules that are produced by a biological organism, including large polymeric molecules such as proteins, polysaccharides, lipids, and nucleic acids (DNA and RNA) as well as small molecules such as primary metabolites, secondary metabolites, and other natural products. For example, in the depicted example, the sample fluid 110 includes DNA molecules 108a and enzymes 108b that interact with the DNA molecules 108a. In various examples, a sample fluid 110 may include various types of biomolecules 108. Moieties of the biomolecules may interact in a biochemical interaction, and aspects, characteristics, or parameters of the biochemical interaction may be determined using a chip-based biosensor 104.

The computing device 114, in the depicted example, implements an analysis module 116. In various examples, a computing device 114 may be a laptop computer, a desktop computer, a smartphone, a handheld computing device, a tablet computing device, a virtual computer, an embedded computing device integrated into an instrument, or the like. In further example, a computing device 114 may communicate with the measurement apparatus 122 via the data network 120. The analysis module 116, in certain examples, is configured to characterize one or more parameters of a biochemical interaction based on measurements of output signals from a biologically gated transistor 106, where the measurements are taken by the measurement apparatus 122.

In the depicted example, the analysis module 116 is separate from the measurement apparatus 122, and is implemented by a computing device 114 separate from the measurement apparatus 122. In another example, the analysis module 116 may be partially or fully integrated with the measurement apparatus 122. For example, the measurement apparatus 122 may include special-purpose logic hardware and/or a processor executing code stored in memory to implement all or part of the analysis module 116. In some examples, the analysis module 116 may be implemented as an embedded processor system or other integrated circuits that form part of a chip-based biosensor 104 and/or part of a chip reader device 102. In some examples, where an analysis module 116 is integrated with the measurement apparatus 122, a system 100 may omit a separate computing device 114.

The remote data repository 118, in various examples, may be a device or set of devices remote from the measurement apparatus 122 and capable of storing data. For example, the remote data repository 118 may be, or may include, a hard disk drive, a solid-state drive, a drive array, or the like. In some examples, the remote data repository 118 may be a data storage device within the computing device 114. In some examples, a remote data repository 118 may be network attached storage, a storage area network, or the like.

In some examples, the measurement apparatus 122 (e.g., a chip-based biosensor 104 and/or a chip reader device 102) may include communication circuitry that transmits measurement information to the remote data repository 118. Measurement information may be measurements from biologically gated transistors 106, or information about the measurements, such as calculated quantities based on the raw measurements. The analysis module 116 may communicate with the remote data repository 118 to characterize one or more parameters of a biochemical interaction based on the information stored by the remote data repository 118. In further examples, the analysis module 116 may store analysis results to the remote data repository 118. In another example, however, the analysis module 116 may receive measurement information from the measurement apparatus 122 directly or over the data network 120, and a remote data repository 118 may be omitted (e.g., in favor of local data storage).

FIG. 2 is a schematic block diagram illustrating one example of an apparatus 200 for excitation and measurement of biochemical interactions, including one example of a biologically gated transistor 106a, coupled to a measurement apparatus 122. The biologically gated transistor 106a is depicted in a top view. The biologically gated transistor 106a and the measurement apparatus 122 in the depicted example may be substantially as described above with reference to FIG. 1, and are described further below.

The biologically gated transistor 106a, in the depicted example, includes a source 212, a drain 202, a channel 210, a reference electrode 208, a counter electrode 204, and a liquid well 206, which are described below. In general, in various examples, a biologically gated transistor 106a may include at least one channel 210 capable of conducting an electrical current between the source 212 and the drain 202. As in an insulated-gate field-effect transistor, current between the source 212 and the drain 202 depends not only not only on a voltage difference between the source 212 and the drain 202 but on certain conditions that affect the conductivity of the channel 210. However, an insulated-gate field-effect transistor is a solid-state device where a gate electrode is separated from the channel by a thin dielectric layer, so that the channel conductivity is modulated by the gate-to-body (or gate-to-source) voltage. Conversely, in various examples, channel conductivity (and a resulting drain-to-source current) for a biologically gated transistor 106a, may be modulated, gated, or affected by liquid-state events. In particular, a sample fluid 110 may be applied to the biologically gated transistor 106a in contact with the channel 210, so that the channel conductivity depends on (or is gated or modulated by) a biochemical interaction of moieties within the sample fluid 110.

In various examples, the source 212, the drain 202, a channel 210, a reference electrode 208, a counter electrode 204, may be formed on a substrate (not shown), such as an oxide or other dielectric layer of a silicon wafer or chip. Certain components of the biologically gated transistor 106a may be formed to be in contact with a sample fluid 110. For example, upper surfaces of the channel 210, the reference electrode 208 and the counter electrode 204 may be exposed or bare for direct interaction with the sample fluid 110. Other components may be covered or electrically insulated from the sample fluid 110. For example, the source 212 and drain 202 may be covered by an insulating layer such as silicon dioxide, silicon nitride, or another dielectric, so that current flows between the source 212 and drain 202 through the channel 210, without the sample fluid 110 creating a short circuit or an alternative or unintended current path between the source 212 and drain 202.

The liquid well 206 may be a structure to contain the sample fluid 110 in a region above the other components of the biologically gated transistor 106a. For example, the liquid well 206 may be a ridge of epoxy, a thermosetting resin, a thermoplastic, or the like. The liquid well 206 may be deposited on the substrate, formed as an opening in the chip packaging for the biologically gated transistor 106a, or the like.

The channel 210, in some examples, is made of a highly sensitive conducting material such as graphene. In further examples, a graphene channel 210 may be deposited on the substrate for the biologically gated transistor 106a by chemical vapor deposition (CVD). In some examples, the channel 210 may be made from another two-dimensional material which has strong in-plane covalent bonding and weak interlayer interactions. Such materials may be referred to as van der Waals materials. For example, in various examples, a channel 210 may be made from graphene nanoribbons (GNR), bilayer graphene, phosphorene, stanine, graphene oxide, reduced graphene, fluorographene, molybdenum disulfide, topological insulators, or the like. Various materials that conduct and exhibit field-effect properties, and are stable at room temperature when directly exposed to various solutions, may be used in a biologically gated transistor 106a. In various implementations, using a biologically gated transistor 106a with one or more channels 210 formed from planar two-dimensional van der Waals materials improves manufacturability, and lowers costs compared with one-dimensional alternatives, such as carbon nanotubes.

The source 212 and drain 202 are disposed at opposite ends of the channel 210 so that a current conducted through the channel 210 is conducted from the drain 202 to the source 212, or from the source 212 to the drain 202. In various examples, the source 212 and drain 202 may be made of conductive material such as gold, platinum, polysilicon, or the like. In some examples, the source 212 may be coupled to the substrate of the biologically gated transistor 106a (e.g., the silicon below the oxide or other dielectric layer) so that a programmable bias voltage (or other programmable bias signal) applied to the source 212 also biases the substrate under the channel 210. In another example, a biologically gated transistor 106a may include a separate body terminal (not shown) for biasing the substrate.

The terms “source” and “drain” may be used herein to refer to conductive regions or electrodes that directly contact the channel 210, or to leads, wires or other conductors connected to those regions or electrodes. Additionally, the terms “source” and “drain” are used as the conventional names for terminals of a transistor, but without necessarily implying a type of charge carrier. For example, a graphene channel 210 may conduct electricity with electrons or holes as the charge carriers depending on various external conditions (such as the biochemical interaction occurring in the sample fluid 110 and the excitation conditions applied by the measurement apparatus 122), and the charge carriers may flow from the source 212 to the drain 202, or from the drain 202 to the source 212.

In various examples, one or more output signals from the biologically gated transistor 106a may be affected by excitation conditions and by a biochemical interaction of moieties within a sample fluid 110. As defined above, the excitation conditions may be physical, electrical, or chemical conditions applied to the biologically gated transistor 106a. Excitation conditions such as programmable bias voltages (or signals), temperature conditions, or the like may be applied to the biologically gated transistor 106a or to the sample fluid 110 by the measurement apparatus 122. The biochemical interaction of moieties within the sample fluid 110 may involve moieties that were within the fluid 110 (e.g., in solution or suspension) when the fluid 110 was prepared, or moieties on the surface of the channel 210, which are within the fluid 110 once the fluid 110 is applied in contact with the channel surface. The biochemical interaction may gate or modulate the channel conductivity, affecting one or more output signals. The output signals may be, or may include, a channel current, a voltage, a capacitance, inductance, or resistance (calculated based on applied and measured voltages and currents), a complex-valued impedance, a complex impedance spectrum, an electrochemical impedance spectrum, a Dirac voltage, a power spectral density, one or more network parameters (such as S-parameters or h-parameters), or the like.

In some examples, certain biomolecules or moieties may be immobilized or functionalized to the surface of the channel 210 to react with other biomolecules or moieties that may be present in the sample fluid 110. For example, the channel 210 may be functionalized with streptavidin to bind with biotinylated molecules in the sample fluid 110. As further examples the channel 210 may be functionalized with antibodies, streptavidin, biotin, neutravidin, avidin, captavidin, zinc finger protein, CRISPR Cas family enzymes, nucleic acids, and synthetic nucleic acid analogs such as peptide nucleic acid, xeno nucleic acid, or the like.

In another example, however, a channel 210 may be bare or unfunctionalized graphene (or include another non-biological material such as a hydrogel or polymer) and may be sensitive to interactions of biomolecules or moieties in the sample fluid 110. For example, in some examples, a channel 210 may be bare or unfunctionalized, but magnetic or non-magnetic particles in the range of about 1 nm to 10 μm in diameter (which may be referred to as “beads”) may be functionalized with streptavidin, biotin, or another material as described above for a functionalized channel 210 and added to the sample fluid 110. Output signals from the biologically gated transistor 106a may be sensitive to interactions between the beads and other molecules or moieties in the sample fluid 110. With magnetic beads, a magnetic field may be applied to attract the beads towards the channel 210 out of the bulk solution of the sample fluid 110, so that the output signals are more strongly affected by the beads in proximity to the channel 210. In other examples, certain reagents such as streptavidin, CRISPR-Cas family enzymes, or the like may be added directly to the sample fluid 110, and output signals may be sensitive to interactions between moieties in the sample fluid 110 even when those moieties are not immobilized to the channel 210.

In some field-effect biosensors using a graphene channel, channel conductivity (and output signals from the biosensor such as currents, capacitances, or the like) may only be significantly responsive to interactions happening at or near the channel surface (e.g., within a double layer and/or a Donnan equilibrium region). Biomolecules or moieties within an electrostatic screening distance (e.g., within a double layer or Donnan equilibrium region) may act as a “screen” between the surface of a channel 210 and the bulk of the sample fluid 110. However, certain biochemical events in the sample fluid 110 away from the channel surface may have a characteristic resonance frequency, corresponding to a physical or chemical motion of biomolecules or moieties. For example, a CRISPR Cas enzyme may repeatedly attach to and cleave DNA substrate molecules at a characteristic frequency. Similarly, a linker molecule that links between two other molecules or moieties (such as an antibody that links an antigen at the Fab region of the antibody to another molecule at the Fc region of the antibody) may act as a spring with a characteristic resonance. Using a measurement apparatus 122 to apply excitation conditions and/or measure output signals for the biologically gated transistor 106a in a frequency bandwidth that includes these characteristic frequencies may allow an apparatus 200 to “see” or detect aspects of the biochemical interaction via detection of a resonance effect, even in the bulk sample fluid 110 outside of the electrostatic screening distance.

Additionally, in some examples, moieties in the bulk sample fluid 110 away from the channel surface may be free to move or interact more quickly (e.g., at higher frequencies) than the ions, molecules, or moieties that are attracted or immobilized to the channel 210 in a double layer or a Donnan equilibrium region. Thus, using a measurement apparatus 122 to apply excitation conditions and/or measure output signals for the biologically gated transistor 106a at frequencies too high for the ions, molecules, or moieties in the double layer or the Donnan equilibrium region to significantly respond may allow an apparatus 200 to “see” or detect aspects of the biochemical interaction in the bulk sample fluid 110 outside of the electrostatic screening distance.

Similarly, the effective screening distance of an ionic double layer may be increased by applying a high-frequency voltage to take advantage of the frequency dependence of the dielectric formed by an ion containing solution, so that the apparatus detects aspects of the biochemical interaction that are within the frequency dependent dynamic interaction distance of the surface, but are outside of the equilibrium electrostatic screening distance. Various excitation conditions and/or measurements of output signals are described in further detail below with reference to subsequent figures.

Accordingly, while some field-effect biosensors rely on moieties immobilized to a channel surface because they can only detect interactions at or near the channel surface, a biologically gated transistor 106a used with a measurement apparatus 122 as disclosed herein may, in some examples, use a bare or unfunctionalized channel surface, because it is sensitive to biochemical interactions occurring in the bulk sample fluid 110 further away from the channel surface. In some examples, a measurement apparatus 122 and a biologically gated transistor 106a with a bare or unfunctionalized channel surface may be used to perform a variety of tests, without requiring different biosensors to be prepared in advance for different tests (e.g., by functionalizing channels in different ways for different tests). In other examples, however, a biologically gated transistor 106a may include a functionalized channel 210, a plurality of channels 210 which may be homogeneously or heterogeneously functionalized, or the like. Various excitation conditions and/or measurements of output signals are described in further detail below with reference to subsequent figures.

In various examples, a liquid (e.g., the sample fluid 110) applied to the channel 210 may be referred to as a liquid gate for the biologically gated transistor 106a, because one or more of the output signals for the biologically gated transistor 106a are affected by conditions, such as a biochemical interaction, within the liquid gate. In addition, in various examples, a biologically gated transistor 106a may include one or more gate electrodes for detecting and/or adjusting a voltage or electric potential of the liquid gate. For example, in the depicted example, the biologically gated transistor 106a includes a reference electrode 208 for measuring an electrochemical potential of the sample fluid 110, and a counter electrode 204 for adjusting the electrochemical potential of the sample fluid 110.

In some examples, an electric potential may develop at the interface between the sample fluid 110 and the reference electrode 208 and/or the counter electrode 204. Thus, in some examples, a reference electrode 208 may be made of a material with a known or stable electrode potential. In another example, however, a reference electrode 208 may be a pseudo-reference electrode that does not maintain a constant electrode potential. Nevertheless, measurements of the electrochemical potential of the sample fluid 110 via a pseudo-reference electrode may still be useful as output signals or as feedback for adjusting the electrochemical potential of the sample fluid 110 via the counter electrode 204. In some examples, the reference electrode 208 and/or the counter electrode 204 may be made of non-reactive materials such as gold or platinum.

In some examples, a pseudo-reference electrode 208 on a chip-based biosensor may be supplemented or replaced by an off-chip reference electrode, which may be an electrochemical reference electrode such as a silver/silver-chloride electrode, a standard calomel electrode, or the like. An off-chip reference electrode may be used in a feedback loop with the on-chip counter electrode 204 to provide more precise and accurate measurement (and control) of the electrochemical potential of the sample fluid 110 than by using an on-chip pseudo-reference electrode 208. Nevertheless, in some examples, the lower level of accuracy and precision provided by the on-chip pseudo-reference electrode 208 may be sufficient for measurement or characterization of certain biochemical interactions.

In some examples, a static or stable potential, provided by stable chemistry at the interface between a reference electrode 208 and the sample fluid 110, may facilitate measuring the voltage of the fluid 110 using the reference electrode 208. For a standard (redox-based) reference electrode, an electrochemical cell produces a known and stable potential via a redox reaction at the reference electrode surface. That cell is connected to the sample fluid such that ions can be exchanged between the cell and the test liquid. This ion exchange leads to a largely resistive impedance between the sample fluid and the reference electrode. The potential of the reference electrode then is adjusted by the potential of the sample fluid.

By contrast, when using an on-chip pseudo-reference electrode 208 made from platinum or another non-reactive material to measure the voltage of the sample fluid 110, there may be no redox reaction at the electrode surface and a largely capacitive impedance across the electrode/liquid interface. There may be a potential drop across this interface, particularly at low frequencies, with the result that the potential of the electrode 208 does not match the potential of the fluid 110. However, this potential drop can be minimized by using excitation circuitry to apply an AC gate voltage to pass AC current through the interface. The on-chip pseudo-reference electrode 208 will be at approximately the potential of the fluid 110 if the interface impedance is small compared to the input resistance of measurement circuitry for monitoring the reference electrode 208 voltage. The interface impedance is given by 1/(2πfC), where f is the frequency of the applied AC current and C is the capacitance at the interface with the reference electrode 208.

However, due to the inverse relationship between interface capacitance and impedance, decreasing the interface capacitance may increase the interface impedance, so that the potential of the electrode 208 does not match the potential of the fluid 110. Contamination of a platinum or non-reactive pseudo-reference electrode 208 may disrupt measurements by decreasing the interface capacitance, or by causing unwanted faradaic currents. Accordingly, in some examples, a protective layer may be provided to avoid contamination of the reference electrode 208 and/or the counter electrode 204. A protective layer may be a material that does not react with or alloy with a platinum reference electrode 208 and/or counter electrode 204, and that can be removed from the reference electrode 208 and/or counter electrode 204 prior to use. For example, aluminum oxide and/or various polymers may be suitable for protection of the reference electrode 208 and/or the counter electrode 204. A user of a biologically gated transistor 106 may remove this protective material prior to use, or a manufacturer may remove this protective material prior to packaging a chip-based biosensor 104 in other packaging that prevents contamination.

In some examples, a biologically gated transistor 106a may be made using photolithography or other commercially available chip fabrication techniques. For example, a thermal oxide layer may be grown on a silicon substrate, and metal components such as a source 212, drain 202, reference electrode 208 and/or the counter electrode 204 may be deposited or patterned on the thermal oxide layer. A graphene channel 210 may be formed using chemical vapor deposition. The use of conventional fabrication techniques may provide low-cost biologically gated transistors 106a, especially in comparison to sensors using high-cost materials such as carbon nanotubes or specialty fabrication techniques. Certain configurations of biologically gated transistors 106a and ways to fabricate and/or improve the sensitivity, reliability, and/or yield of various biologically gated transistors 106a are discussed in U.S. patent application Ser. No. 15/623,279 entitled “PATTERNING GRAPHENE WITH A HARD MASK COATING”; U.S. patent application Ser. No. 15/623,295 entitled “PROVIDING A TEMPORARY PROTECTIVE LAYER ON A GRAPHENE SHEET”; U.S. patent application Ser. No. 16/522,566 entitled “SYSTEMS FOR TRANSFERRING GRAPHENE”; and U.S. Pat. No. 10,395,928 entitled “DEPOSITING A PASSIVATION LAYER ON A GRAPHENE SHEET”; each of which is incorporated herein by reference.

FIG. 3 is a schematic block diagram illustrating another example of an apparatus 300 for excitation and measurement of biochemical interactions, including another example of a biologically gated transistor 106b, coupled to a measurement apparatus 122. As in FIG. 2, the biologically gated transistor 106b is depicted in a top view. The biologically gated transistor 106b and the measurement apparatus 122 in the depicted example may be substantially as described above with reference to FIGS. 1 and 2, and are described further below.

In the depicted example, the biologically gated transistor 106b includes a source 312, a plurality of drains 302, a plurality of channels 210, a reference electrode 308, and a counter electrode 304, which may be substantially similar to the source 212, drain 202, channel 210, reference electrode 208, and counter electrode 204 described above with reference to FIG. 2. (A liquid well similar to the liquid well 206 of FIG. 2 is not depicted in FIG. 3 but may similarly be provided as part of the biologically gated transistor 106b)

However, in the depicted example, the biologically gated transistor 106b includes a plurality of channels 310, and a plurality of drains 302. In various examples, a plurality of channels 310 may be homogeneous or heterogeneous. For example, homogeneous channels 310 may be bare or unfunctionalized graphene, or may be functionalized in the same way. Conversely, heterogeneous channels 310 may be a mixture of bare and functionalized graphene channels 310, a mixture of channels 310 that are functionalized in more than one way (optionally including one or more unfunctionalized channels 310) or the like. In some examples, providing a plurality of heterogeneous channels 310 may make a biologically gated transistor 106b useful for a variety of different tests that rely on events near the surfaces of the channels 310.

However, in some examples, a measurement apparatus 122 may obtain information about aspects of the biochemical interaction occurring at measurement distances greater than the electrostatic screening distance (e.g., in the bulk sample fluid 110, outside the double layer or a Donnan equilibrium region). The measurement bandwidth used by the measurement apparatus 122 may correspond to one or more measurement distances greater than the electrostatic screening distance, and tests or measurements using the biologically gated transistor 106b may be made without functionalizing the surfaces of graphene channels 310. This approach could also be used to probe the properties of the Donnan capacitance. Even with bare or unfunctionalized channels 310, the use of multiple channels 310 may provide redundancy to mitigate damage to any individual channel 310 (e.g., mechanical damage from a pipette tip used to apply the sample fluid 110), and may make the biologically gated transistor 106b sensitive to biochemical interactions in the sample fluid 110 across a greater surface area than in a single-channel device.

In some examples, a biologically gated transistor 106b may include a plurality of drains 302 coupled to the channels 310. In various examples, one drain 302 may be provided per channel 310 so that each channel 310 can be independently biased. In certain embodiments, however, channels 310 may be coupled to drains 302 in groups, so that the channels 310 of a group can be biased together in parallel, but different groups can be biased differently. For example, in the depicted example, the biologically gated transistor 106b includes fifteen channels 310, coupled to three drains 302a-c, so that one of the drains 302 can be used to bias a group of five channels 310. In one or more examples, a plurality of channels 310 may be coupled in parallel to a single drain 302.

In the depicted example, the channels 310 are coupled in parallel to one source 312. For some measurements, the source 312 may be coupled to ground (e.g., 0 volts, or another reference voltage). In one or more examples, the channels 310 may be coupled to a plurality of sources 312, allowing different measurements to be made with different source biases. For example, channels 310 may be coupled to multiple sources 312 individually or in groups, as described above for the plurality of drains 302.

Functionalization of transistor channels 310, in some examples, may include applying different voltages to different channels 310 to attract or repel different charges. For example, to heterogeneously functionalize channels 310 of the biologically gated transistor 106b, a solution may be applied to the transistor 106b with a target functionalization chemistry to be attached to the channels 310 coupled to drain 302a. If that target chemistry is negatively charged in solution, a voltage may be applied to drain 302a to attract negative charges to the channels 310 coupled to drain 302a, while another voltage may be applied to drains 302b, 302c to repel negative charges away from the channels 310 coupled to those drains.

With a subset of channels 310 thus functionalized with the target functionalization chemistry, the solution may be removed, and another solution may be applied with a different target functionalization chemistry to be attached to the channels 310 coupled to drain 302b. Similarly, a voltage may be applied to drain 302b to attract the target functionalization chemistry, with another voltage applied to other drains 302a, 302c to repel the target functionalization chemistry.

By applying a positive or negative voltage to a channel 310 to attract or repel a positively or negatively charged molecule or moiety used for functionalization, the voltage controls whether (or to what extent) the channel is functionalized by the solution. Thus, solutions for functionalizing transistor channels 310 may be applied to and removed from a multi-channel transistor or an array of transistors in sequence, using a liquid handler or a simple flow cell instead of more complex microfluidics or precise micro-droplet positioning, while voltage control of the channels 310 is used to determine which channels are functionalized by which solutions. For example, if there are multiple transistors 106 on a chip-based biosensor 104, each transistor may be functionalized differently in turn by applying and removing different solutions, and for each solution that is applied, using voltage control of the transistor channels to attract the desired chemistry to one transistor while repelling it from the other transistors in the array.

In the depicted example, the reference electrode 308 and the counter electrode 304 are disposed so that the channels 310 are between the reference electrode 308 and the counter electrode 304. In this configuration, the electrochemical potential of the liquid gate may be modified via the counter electrode 304 and monitored via the reference electrode 308, so that the electrochemical potential near the channels 310 is close to the modified and/or monitored potential. Additionally, in the depicted example, the counter electrode 304 is significantly larger than the channels 310 or the reference electrode 308, so that modifications to the electrochemical potential of the liquid gate made via the counter electrode 304 quickly occur across a large surface area, and in a large volume of the sample fluid 110.

Although FIGS. 2 and 3 depict individual biologically gated transistors 106a, 106b, a chip-based biosensor 104 in various examples may include a plurality of biologically gated transistors 106, which may be homogeneously or heterogeneously configured. For example, the homogeneous or heterogeneous configurations described above for multiple channels 310 in one biologically gated transistor 106b may similarly apply to multiple biologically gated transistors 106, each with their own independent source, drain, reference, and counter terminals.

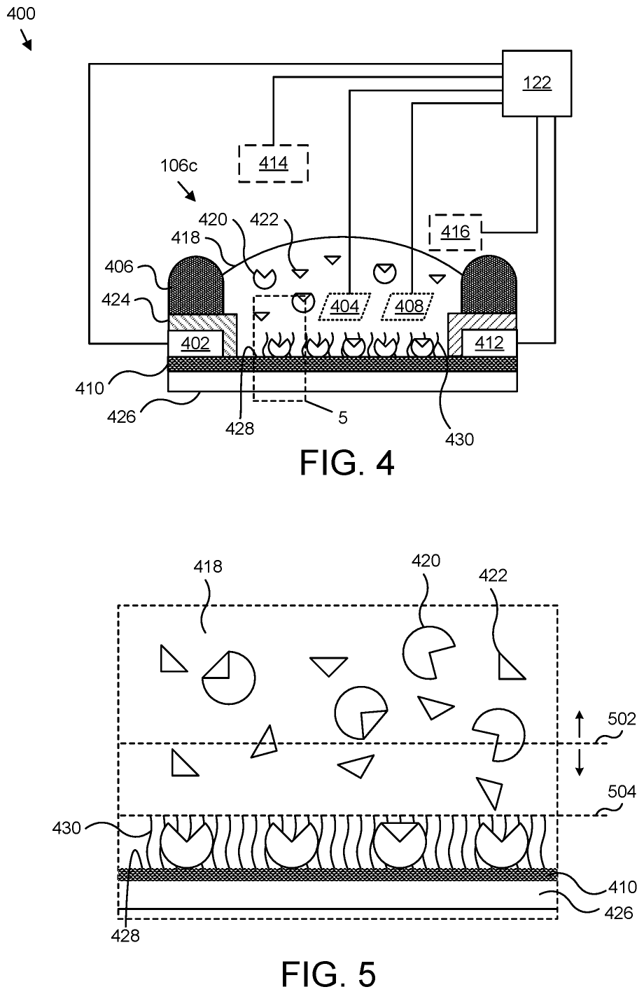

FIG. 4 is a schematic block diagram illustrating a further example of an apparatus 400 for excitation and measurement of biochemical interactions, including a further example of biologically gated transistor 106c, coupled to a measurement apparatus 122. The biologically gated transistor 106c is depicted in a cross-section view, from the side. The biologically gated transistor 106c and the measurement apparatus 122 in the depicted example may be substantially as described above with reference to FIGS. 1 through 3, which are described further below.

In the depicted example, the biologically gated transistor 106b includes a source 412, a drain 402, a channel 410, a reference electrode 408, a counter electrode 404, and a liquid well 406, which may be substantially as described above. The channel 410, in the depicted example, is a two-dimensional graphene region disposed on a dielectric layer 426 above a substrate (not shown). The source 412 and drain 402 are formed in contact with the channel 410, and are covered by a dielectric 424 (e.g., silicon nitride). A sample fluid 418 (which may be substantially similar to the sample fluid 110 described above) is applied in contact with the surface 428 of the channel 410. For example, the sample fluid 418 may be pipetted (or otherwise inserted) into the liquid well 406 to contact the channel surface 428, the reference electrode 408, and the counter electrode 404. The dielectric 424 electrically insulates the source 412 and drain 402 from the sample fluid 418, so that current between the source 412 and drain 402 is through the channel 410 rather than directly through the sample fluid 418.

In the depicted example, the sample fluid 418 includes a plurality of biomolecules or moieties 420, 422. Biomolecules 420 (e.g., proteins) are represented by circular sections, and moieties 422 that interact with proteins (e.g., antigens for antibody proteins, substrates for enzyme proteins, or the like) are represented by triangles. Thus, in the depicted example, a biochemical interaction may occur between proteins 420 and other moieties 422. Additionally, although a protein-based interaction is depicted, various other or further types of moieties and types of interactions of moieties may occur in s a sample fluid 418.

In the depicted example, the surface 428 of the channel 410 is functionalized by immobilization of certain moieties 420 to the channel surface 428. A blocking layer 430, represented by curved lines, may immobilize moieties to the surface. In various examples, a blocking layer 430 may include polyethylene glycol or other molecules or polymers capable of binding ions, molecules, or moieties to the surface 428. The blocking layer 430 may be permeable to certain other ions, molecules, or moieties in the sample fluid 418. For example, the blocking layer 430 may bind proteins 420 to the surface 428 but may be permeable to the moieties 422 that interact with the proteins 420. Although the surface 428 of the channel 410 is functionalized in the depicted example, a channel 410 in another example may be a bare or unfunctionalized channel, without moieties that are immobilized to the surface 428 (e.g., without a blocking layer 430).

The measurement apparatus 122, in the depicted example, is coupled to the source 412, the drain 402, the reference electrode 408, and the counter electrode 404. In various examples, the measurement apparatus 122 may apply excitation conditions to the biologically gated transistor 106c via the source 412, the drain 402, and/or the counter electrode 404. In further examples, the measurement apparatus 122 may perform measurements of one or more output signals from the biologically gated transistor 106c via the source 412, the drain 402, and/or the reference electrode 408.

In some examples, an apparatus 400 may include temperature control circuitry 414, and/or a fluidic device 416. The measurement apparatus 122 may include or communicate with the temperature control circuitry 414, and/or a fluidic device 416, and may control the temperature control circuitry 414, and/or fluidic device 416. FIG. 4 depicts the temperature control circuitry 414 and a fluidic device 416 in dashed lines, indicating that they may be present in some examples or absent in other examples.

In various examples, the measurement apparatus 122 may control a temperature of the sample fluid 418 using temperature control circuitry 414 for various reasons, such as to determine how a biochemical interaction occurs at a predetermined temperature (such as body temperature) or to see how one or more aspects of a biochemical interaction changes in response to heating or cooling. Temperature control circuitry 414, in various examples, may be any circuitry configured to control the temperature or operable to change the temperature of the sample fluid 418 and/or the biologically gated transistor 106c. In some examples, temperature control circuitry 414 may be capable of heating the sample fluid 418 and/or the biologically gated transistor 106c. In some examples, temperature control circuitry 414 may be capable of cooling the sample fluid 418 and/or the biologically gated transistor 106c. In some examples, temperature control circuitry 414 may be provided for both heating and cooling.

In various examples, temperature control circuitry 414 may include components such as a resistive heater in proximity to the chip-based biosensor 104, a resistive wire on the same substrate as the biologically gated transistor 106c, a Joule heating controller to control the current in a resistive element (or in the channel 410 itself, used as a resistive element for Joule heating), a solid-state heat pump (e.g., using the Peltier effect). In some examples, temperature control circuitry 414 may include components for monitoring the temperature of the sample fluid 418 and/or the biologically gated transistor 106c (and for controlling the temperature based on the monitored temperature), such as a thermistor, one or more thermocouples, a silicon bandgap temperature sensor, a resistance thermometer, or the like. Various other or further components for measuring or controlling a temperature may be included as temperature control circuitry 414 in various examples of an apparatus 400 or a measurement apparatus 122.

In some examples, one or more fluidic devices 416 may be used to drive sample flow through a flow cell or other fluidic or microfluidic channels. In various examples, the biologically gated transistor 106c may use a flow cell. However, in certain examples, the biologically gated transistor 106c may be highly sensitive and may perform high-sensitivity measurements without a flow cell. In some examples, a chip-based biosensor 104 may include multiple biologically gated transistors 106c, and a fluidic device 416 may drive flow of a sample fluid over a sequence of biologically gated transistors 106c so that upstream and downstream transistors are, respectively, sensitive to earlier and later aspects of a biochemical interaction occurring at different times.

In various examples, the measurement apparatus 122 may apply one or more excitation conditions to the biologically gated transistor 106c, so that one or more output signals from the biologically gated transistor 106c are affected by the excitation conditions and by the biochemical interaction of moieties 420, 422 in the sample fluid 418. In further examples, the measurement apparatus 122 may obtain information that corresponds to aspects or portions of the biochemical interaction occurring at one or more measurement distances from the surface 428 of the channel 410, by performing time-dependent measurements of at least one of the output signals. Measurement distance and other distances relative to the surface 428 are described in further detail below with reference to FIG. 5.

FIG. 5 is a detail view of a region outlined in dashed lines in FIG. 4, and depicts a measurement distance 502, and an electrostatic screening distance 504. Portions of the channel 410, channel surface 428, dielectric layer 426, blocking layer 430, and the sample fluid 418 (including moieties 420, 422) described above with reference to FIG. 4 are also depicted. The measurement distance 502 and the electrostatic screening distance 504 are indicated by dashed lines at the respective distances from the channel surface 428. For example, the measurement distance 502 is indicated by a dashed line where points on the line are a measurement distance 502 away from the channel surface 428

The measurement distance 502, in the depicted example, is a distance from the channel surface 428 such that the effect of at least some aspect or portion of a biochemical interaction occurring at the measurement distance 502, on an output signal of the biologically gated transistor 106, is detectable by the measurement apparatus 122. Whether an effect on an output signal is detectable by the measurement apparatus 122 may be relative to noise, interference from other events affecting the same output signal, a predetermined detection threshold, or the like. For example, a protein 420 binding to moiety 422 may detectably affect an output signal if the binding happens at or within the measurement distance 502 from the channel surface 428, but may not detectably affect the output signal if the binding happens further than a measurement distance 502 away from the channel surface 428.

In various examples, the measurement distance 502 may depend on or correspond to excitation conditions applied by the measurement apparatus 122, or to a measurement frequency or bandwidth. For example, moieties immobilized to the channel surface 428 (e.g., in the blocking layer 430) may not be able to move or interact quickly in response to high frequency excitation (of high-frequency components of broadband excitation, thermal molecular movements, or the like). Thus, measurement using a bandwidth or frequency range that includes high frequencies may provide increased measurement distances 502, allowing the measurement apparatus 122 to “see” or detect interactions further away from the channel 410. Conversely, measurement at lower frequencies may detect interactions within a shorter measurement distance 502. In FIG. 5, arrows above and below the dashed line for the measurement distance 502 indicate that the measurement distance 502 may be increased or decreased based on excitation conditions and/or measurement bandwidth.

The electrostatic screening distance 504, in various examples, may be a measurement distance for steady state or low frequency measurements (e.g., at frequencies under 10 Hz). Under steady-state or low frequency conditions (e.g., if higher-frequency interactions are not excited and/or not measured), output signals may only be detectably affected by aspects or portions of the biochemical interaction occurring near the channel surface 428. For example, in the depicted example, a Donnan equilibrium region is formed by larger molecules or moieties 420 immobilized to the surface 428 (e.g., in the blocking layer 430). Although higher-frequency excitation and measurement may distinguish faster-moving interactions (or characteristic resonances) outside the Donnan equilibrium region from slower-moving interactions of the immobilized molecules or moieties, output signals for steady-state or low-frequency excitation and measurement may be affected by aspects or portions of the biochemical interaction occurring in the Donnan equilibrium region. Thus, the electrostatic screening distance 504 in the depicted example is based on the thickness of the Donnan equilibrium region, but the measurement distance 502 may be greater than the electrostatic screening distance 504 when the measurement apparatus 122 applies higher-frequency excitation conditions and/or makes higher-frequency measurements.

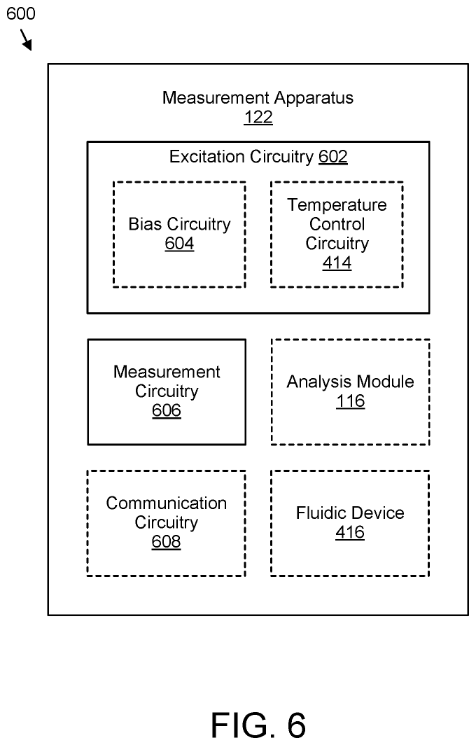

In one or more other examples, an ionic double layer may form in the absence of a Donnan equilibrium region (e.g., if a blocking layer 430 is omitted). As in the Donnan equilibrium region, the ions at or near the surface 428 may screen low-frequency interactions occurring further away from the surface from detectably affecting the output signals, and the electrostatic screening distance 504 may be based on the thickness of the ionic double layer. (In some examples, if an ionic double layer and a Donnan equilibrium region both exist, the electrostatic screening distance 504 may be based on which layer or region is thicker). Thus, as described above for the Donnan equilibrium region, high-frequency measurements may detect events occurring at a measurement distance 502 greater than the electrostatic screening distance 504. Additionally, in some examples, application of changing excitation potentials may move the ions in the double layer, increasing the measurement distance 502 by increasing the double layer thickness as compared to the electrostatic screening distance 504 (e.g., the double layer thickness under steady state or low-frequency excitation conditions). Excitation and measurement circuitry for measurements at measurement distances 502 greater than electrostatic screening distances 504 are described in further detail below with reference to FIG. 6.

FIG. 6 is a schematic block diagram illustrating a further apparatus 600 for excitation and measurement of biochemical interactions, including an instance of a measurement apparatus 122, in accordance with one or more examples of the present disclosure. In the depicted example, the measurement apparatus 122 includes excitation circuitry 602 and measurement circuitry 606. Certain components indicated by dashed lines in FIG. 6 are included in the depicted example, but may be omitted in one or more other examples. In the depicted example, the excitation circuitry 602 includes bias circuitry 604 and temperature control circuitry 414. In the depicted example, the measurement apparatus 122 includes an analysis module 116, communication circuitry 608, and a fluidic device 416. The measurement apparatus 122, temperature control circuitry 414, analysis module 116, and fluidic device 416 in the depicted example may be substantially as described above with reference to previous Figures.

In various examples, the measurement apparatus 122 may use excitation circuitry 602 to apply excitation conditions to a biologically gated transistor 106, and may use measurement circuitry 606 to perform time-dependent measurements of one or more output signals from the biologically gated transistor 106. The output signal(s) may be affected by the excitation conditions, and by a biochemical interaction of moieties within a sample fluid 110 applied to a channel surface 428 for the biologically gated transistor 106. The measurement circuitry 606 may obtain information corresponding to the biochemical interaction at one or more measurement distances 502 greater than an electrostatic screening distance 504, by measuring the output signal(s) using a measurement bandwidth that corresponds to the one or more measurement distances 502.

In some examples, the measurement apparatus 122 may include an analysis module 116 to characterize one or more parameters of the biochemical interaction based on the plurality of time-dependent measurements from the measurement circuitry 606. In some examples, however, the measurement apparatus 122 may not include an analysis module 116. For example, in one or more examples an analysis module 116 may be implemented by a computing device 114 separate from the measurement apparatus 122. In some examples, the measurement apparatus 122 may include communication circuitry 608 to transmit the measurements from the measurement circuitry 606, or information based on the measurements, to a remote data repository 118.

The excitation circuitry 602, in the depicted example, is configured to apply one or more excitation conditions to a biologically gated transistor 106, or a set of biologically gated transistors 106. An excitation condition, in various examples, may be a physical, chemical, or electrical condition applied to biologically gated transistor 106, such as a voltage, amplitude, frequency, amplitude, phase, or waveform for an electrical or electrochemical excitation, a temperature, a fluid flow rate, or the like. Excitation circuitry 602, may be any circuitry that applies, modifies, removes, or otherwise controls one or more excitation conditions.

In some examples, excitation conditions may include one or more programmable biases applied to a biologically gated transistor 106, and excitation circuitry 602 may use bias circuitry 604 to control, vary, modulate, and/or apply the programmable biases. A programmable bias, in various examples, may be an electrical signal or waveform, such as a constant voltage or current selected by bias circuitry 604, a square wave, a sine wave, a more complicated waveform such as a sum of sine waves of various amplitudes, frequencies and phases (possibly also including a zero-frequency or DC offset component), or the like. In various examples, programmable biases may include a source bias applied to a source 212 of the biologically gated transistor 106, a drain bias applied to a drain 202 of the biologically gated transistor 106, and/or a gate bias applied to a liquid gate of the biologically gated transistor 106 (e.g., applied to a sample fluid 110 in contact with the channel 210 of the transistor 106 via a counter electrode 204, and possibly controlled based on feedback from a reference electrode 208).

A source bias, in some examples, may be zero volts, ground or another DC reference voltage. For example, the source 212 may be connected to ground, so that gate-to-source and drain-to-source voltage differences can be simplified to a gate bias and a drain bias. However, in some examples, a source bias may be a programmable bias other than zero volts or ground. For example, the bias circuitry 604 may vary the source bias over time in a sweep, a waveform, or the like. In further examples, the bias circuitry 604 may vary, sweep, or modulate the source bias, the gate bias, and/or the drain bias.

Bias circuitry 604 for controlling, varying, modulating, and/or applying programmable biases to a biologically gated transistor 106, in various examples, may include any circuitry capable of generating or modulating programmable biases, such as power supplies, voltage sources, current sources, oscillators, amplifiers, function generators, bias tees (e.g., to add a DC offset to an oscillating waveform), a processor executing code to control input/output pins, signal generation portions of source measure units, lock-in amplifiers, network analyzers, chemical impedance analyzers, or the like. Bias circuitry 604 in various other or further examples may include various other or further circuitry for creating and applying programmable biases.

In some examples, programmable biases may be electrical potentials applied via the source 212 and drain 202 terminals of a biologically gated transistor 106. In some examples, a programmable bias may be an electrochemical potential. For example, in one example, the bias circuitry 604 is configured to adjust the electrochemical potential of the sample fluid 110 by varying a voltage applied to a counter electrode 204 of the biologically gated transistor 106.

In some examples, excitation conditions may include a temperature for the sample fluid 110 applied to a biologically gated transistor 106, and excitation circuitry 602 may use temperature control circuitry 414 to control the temperature. Controlling the temperature, in various examples, may include increasing or decreasing the temperature (e.g., to detect or analyze temperature-sensitive aspects of a biochemical interaction) maintaining a temperature in a range or near a target temperature, monitoring temperature for feedback-based control, or the like. Thus, as described above, temperature control circuitry 414 may include any circuitry capable of changing the temperature of the sample fluid 110 and/or the biologically gated transistor 106. For example, in various examples, temperature control circuitry 414 may include a resistive heater, a Joule heating controller to control current in a resistive heater (or in the channel 210 itself), a solid-state heat pump, a thermistor, or the like. Temperature control circuitry 414 in various other or further examples may include various other or further circuitry for controlling or measuring a temperature.

Additionally, in some examples, excitation circuitry 602 may include circuitry other than or in addition to bias circuitry 604 and/or temperature control circuitry 414, for applying excitation conditions other than or in addition to programmable biases and/or temperature. For example, excitation circuitry 602 may include electromagnets for magnetic excitation, light emitters of any desired wavelength, radioactive sources, emitters of ultraviolet light, x-rays, gamma rays, electron beams, or the like, ultrasonic transducers, mechanical agitators, or the like. Various other or further types of excitation circuitry 602 may be used to apply various other or further excitation conditions.

As described above, one or more output signals for a biologically gated transistor 106 may be affected by or sensitive to one or more of the excitation conditions applied by the excitation circuitry 602 and by a biochemical interaction of moieties within the sample fluid 110, in contact with the channel surface 428. As a simple example, with excitation conditions that include a constant drain-to-source bias voltage, a biochemical interaction of moieties at or near the channel surface 428 may affect an output signal, such as a drain-to-source current, a capacitance of an ionic double layer formed at the channel surface 428 (e.g., as measured between the drain 202 and the reference electrode 208), or the like. Higher-frequency excitation may affect output signals in various ways as described herein. Various output signals that may be affected by a biochemical interaction, and measured, may include a complex resistance (e.g., impedance) of the channel 210, electrical current through the channel 210, voltage drop across the channel 210, coupling between the channel 210 and the liquid gate (e.g., biased and/or measured via a counter electrode 204 and/or a reference electrode 208), electrical (channel) and/or electrochemical (liquid gate) voltages, currents, resistances, capacitances, inductances, complex impedances, network parameters (e.g., S-parameters or h-parameters determined using a network analyzer), a Dirac voltage (e.g., a liquid gate voltage that minimizes channel current in a graphene channel 210), charge carrier mobility, contact resistance, kinetic inductance, a spectrum based on multiple measurements such as a power spectral density, an electrical impedance spectrum, an electrochemical impedance spectrum, or the like.

Because certain output signals from the biologically gated transistor 106 may be affected by a biochemical interaction of moieties within the sample fluid 110, information corresponding to the biochemical interaction may be obtained by measuring one or more of the affected output signals. Information corresponding to the biochemical interaction may be information directly about the interaction, or information that affects or is affected by the interaction. For example, information corresponding to the biochemical interaction may be information such as whether or not an interaction occurs under certain conditions, the extent to which a reaction occurs, whether a certain moiety or molecule is present or absent, the concentration of a certain moiety or molecule, information about the interaction in the sample fluid 110 overall, information about the interaction in a portion or regions of the sample fluid 110 (e.g., at or within a particular measurement distance 502), or the like.

Thus, in various examples, the measurement circuitry 606 may be configured to perform a plurality of time-dependent measurements of at least one of the one or more output signals affected by the excitation conditions and the biochemical interaction. Measurement circuitry 606, in various examples, may include any circuitry capable of performing time-dependent measurements of one or more output signals. For example, in some examples, measurement circuitry 606 may include preamplifiers, amplifiers, filters, voltage followers, data acquisition (DAQ) devices or boards, sensor or transducer circuitry, signal conditioning circuitry, an analog-to-digital converter, a processor executing code to receive and process signals via input/output pins, measurement portions of source measure units, lock-in amplifiers, network analyzers, chemical impedance analyzers, or the like. Measurement circuitry 606 various other or further examples may include various other or further circuitry for performing time-dependent measurements of output signals.

In some examples, output signals affected by the excitation conditions and the biochemical interaction may be small in amplitude, and measurement circuitry 606 may include one or more types of amplifiers to amplify the output signals. Amplifier systems or circuits may include operational amplifiers (“op-amps”). However, the gain, noise, and bandwidth of the measurement may be ultimately limited by the op-amp in use. Some amplification circuits may provide a larger signal to noise ratio than others.

In various examples, measurement circuitry 606 may include a transimpedance amplifier, used to measure the transimpedance of the device, which is the change in resistance in response to a change in the surface potential at the channel of a transistor 106. A transimpedance amplifier may be a current to voltage amplifier, with gain set by a feedback resistor. The noise limit for a transimpedance amplifier may correspond to the Norton equivalent circuit source capacitance of the device and wiring.

In certain examples, measurement circuitry 606 may include a source-drain follower circuit for amplification of output signals. A source-drain follower may be a negative feedback op-amp system, which measures the surface potential at the channel of a biologically gated transistor 106 by adjusting the source-gate voltage to maintain a constant drain current.

In various examples, measurement circuitry 606 may include various other or further amplification circuitry to provide a high signal-to-noise ratio for high frequency signals. In some examples, measurement circuitry 606 may include multiple types of amplifiers, to measure multiple signals or parameters simultaneously.

Time-dependent measurements, in various examples, may include a series of measurements taken over time. Thus, for example, time-dependent measurements of an output signal may reveal how the output signal is changing over time (or may reveal whether the output signal is remaining constant). Time dependent measurements may be measurements of electrical and/or electrochemical output signals. For example, in some examples, electrical output signals may be measured via the source 212 and drain 202 terminals of a biologically gated transistor 106. In some examples, the plurality of time-dependent measurements includes measurements of an electrochemical potential of the sample fluid 110 via a reference electrode 208 of the biologically gated transistor 106.

Measurement circuitry 606 may perform time-dependent measurements using a measurement bandwidth, which is (as defined above) a band or range of frequencies for which the output signals are measured. For example, where discrete samples of the output signals are measured at a sampling rate, the measurement bandwidth may be a range from 0 Hz to half the sampling rate. As another example, measurement circuitry 606 may include one or more filters such as low-pass filters, high-pass filters, band-pass filters, notch filters, or the like, and the measurement bandwidth may be determined by which filters are used.

As described above with reference to FIG. 5, lower-frequency components of output signals may be dominated by aspects or portions of the biochemical interaction near the channel surface 428, while higher frequency components of the output signals may reveal aspects or portions of the biochemical interaction further away from the channel surface 428. For example, high-frequency excitation of a biochemical interaction (e.g., by high-frequency waveforms applied by bias circuitry 604 or even by high-frequency components of ambient or thermal noise) may result in motion or interaction of moieties in the bulk of the sample fluid 110, but moieties immobilized to the channel surface 428 may not be able to move or interact quickly in response to high frequency excitation to the same extent. Thus, a frequency within the measurement bandwidth may correspond to a measurement distance 502, so that the spectral component of an output signal, at that frequency, corresponds to the biochemical interaction occurring at or within that measurement distance 502 from the channel surface 428.

Accordingly, in some examples, the measurement circuitry 606 may be configured to obtain information corresponding to the biochemical interaction occurring at one or more measurement distances 502, by using a predetermined measurement bandwidth corresponding to the one or more measurement distances 502. A measurement bandwidth and/or the corresponding measurement distances 502 from the channel surface 428 may be predetermined by a user or a manufacturer of a measurement apparatus 122, and may depend on what aspect of a biochemical interaction is to be observed, or on what distances from the channel surface 428 are of interest. In some examples, at least one of the measurement distances 502 may be greater than the electrostatic screening distance 504 from the channel surface 428.

In various examples, measurement circuitry 606 that performs time-dependent measurements of one or more output signals may “see” or detect information about biomolecular reactions in real time, over the course of the time-dependent measurements. Also, in some examples, measurement circuitry 606 that uses a predetermined measurement bandwidth corresponding to one or more measurement distances 502, with at least one of the measurement distances 502 being greater than the electrostatic screening distance 504, may “see” or detect information about biomolecular reactions in the bulk sample fluid 110 instead of only near the channel surface 428

In various examples, portions or components of excitation circuitry 602 and/or measurement circuitry 606 may be disposed in a chip-based biosensor 104, a chip reader device 102, or in a separate device (e.g., lab bench test and measurement equipment) coupled to the chip-based biosensor 104. For example, single-use components such as a resistive heater component for excitation circuitry 602 may be disposed on a chip-based biosensor 104, while multi-use components such a digital signal processing circuitry for generating or analyzing complex waveforms may be disposed in a chip reader device 102. Various other ways to dispose or arrange portions or components of excitation circuitry 602 and/or measurement circuitry 606 may be used in various other examples.

In some examples, portions or components of excitation circuitry 602 and/or measurement circuitry 606 may be integrated with one or more biologically gated transistors 106 in a chip-based biosensor 104. For example, existing CMOS fabrication techniques may be used to form electronics for excitation circuitry 602 and/or measurement circuitry 606 in a silicon substrate, prior to forming a biologically gated transistor 106 above the excitation circuitry 602 and/or measurement circuitry 606. To integrate a biologically gated transistor 106 with a CMOS patterned wafer, the top of the CMOS wafer may be patterned with a dielectric layer 426 and metal connections in a similar pattern that would be used for a standalone biologically gated transistor 106, but with source 212 and drain 202 electrodes, as well as reference electrodes 208 and counter electrodes 204, connected to CMOS excitation circuitry 602 and/or measurement circuitry 606 under the biologically gated transistor 106.

In some examples, providing excitation circuitry 602 and/or measurement circuitry 606 in a CMOS layer under a biologically gated transistor 106 may eliminate longer traces or wires that would otherwise go between the biologically gated transistor 106 and the excitation circuitry 602 and/or measurement circuitry 606, thus removing noise and complications due to capacitance, antenna effects of the connected wires, and the like. In some examples, providing excitation circuitry 602 and/or measurement circuitry 606 in a CMOS layer under a biologically gated transistor 106 may allow chip-based biosensors 104 to include arrays of biologically gated transistors 106, with integrated excitation circuitry 602 and/or measurement circuitry 606.

The analysis module 116, in some examples, is configured to characterize one or more parameters of the biochemical interaction based on the plurality of time-dependent measurements performed by the measurement circuitry 606. A parameter of the biochemical interaction, in various examples, may be information about the interaction such as whether or not an interaction occurs under certain conditions, a reaction rate, presence, absence or concentration of a molecule or moiety, or the like. Characterizing a parameter of the interaction, in various examples, may include determining a parameter, determining information about a parameter (such as whether a parameter is above or below a threshold value) or the like. In various examples, an analysis module 116 may use various methods, including known quantitative analysis methods to characterize a parameter of a biochemical interaction. Results from the analysis module 116, such as parameters characterized by the analysis module 116, may be communicated to a user directly via a display or printout (e.g., from the chip reader device 102), transmitted to a user via data network 120, saved to a storage medium (e.g., in remote data repository 118) for later access by one or more users, or the like.

In some examples, the analysis module 116 may characterize one or more parameters of the biochemical interaction by determining an observed spectrum based on the plurality of time-dependent measurements and comparing the observed spectrum to one or more model spectra corresponding to one or more models of biochemical interactions. An observed spectrum, in various examples, may be data that relates time-dependent measurements performed by the measurement circuitry 606, or other quantities calculated based on the time-dependent measurements, to a frequency. For example, an observed spectrum may be frequency-domain data obtained by sweeping an excitation frequency for a programmable bias applied to a biologically gated transistor 106, sweeping a measurement frequency across the measurement bandwidth, performing a fast Fourier transform (FFT) (or a related transform such as a Laplace transform or Z-transform) of time-domain data (e.g., the time-dependent measurements performed by the measurement circuitry 606), or the like. Various examples of an observed spectrum may include a power spectral density, a complex-valued electrical impedance spectrum, a complex-valued electrochemical impedance spectrum, or the like.

In some examples, an observed spectrum may be a real-valued function of frequency. For example, a power spectral density may relate real-valued power amplitudes to frequencies. In some examples, an observed spectrum may be a complex-valued function of frequency. For example, an impedance spectrum may have real and imaginary components based on how measured current amplitudes and phases relate to applied voltage amplitudes and phases at different frequencies.

In certain examples, the analysis module 116 may determine an observed spectrum by calculating the observed spectrum based on the time-dependent measurements from the measurement circuitry 606. For example, the analysis module 116 may determine an impedance spectrum based on programmable bias voltages applied by the excitation circuitry 602 and currents measured by the measurement circuitry 606. In one or more examples, however, the analysis module 116 may determine an observed spectrum by receiving the already-calculated observed spectrum from the measurement circuitry 606. For example, the measurement circuitry 606 may sweep a measurement frequency across the measurement bandwidth to produce an observed spectrum, and may communicate the observed spectrum to the analysis module 116.

By contrast, a model spectrum may be a spectrum similar to or corresponding to the observed spectrum, but based on a model of a biochemical interaction. For example, where the observed spectrum is a power spectral density, a model spectrum may be a predicted or modeled power spectral density based on a model of what biochemical interactions may occur in the sample fluid 110. A model spectrum may be a predicted spectrum based on a computer model of a biochemical interaction, or may be an observed spectrum from a known interaction (e.g., previously measured using a sample fluid 110 with known/controlled reagents, moieties, or molecules). A plurality of different model spectra may correspond to different models of what biochemical interactions occur, or may occur, in the sample fluid 110. Thus, in some examples, the analysis module 116 may characterize one or more parameters of a biochemical interaction by comparing the observed spectrum to one or more model spectra. The extent to which an observed spectrum matches a model spectrum may indicate the extent to which the biochemical interaction is similar to a model of a biochemical interaction. Accordingly, the analysis module 116 may characterize a parameter of the interaction, such as which model interaction is most similar, by calculating some measure of similarity such as a cross-correlation, partial correlation, or the like, between the observed spectrum and one or more model spectra, and selecting a model for which the model spectrum is most similar to the observed spectrum.

In some examples, an analysis module 116 may be separate from the measurement apparatus 122. For example, an analysis module 116 may be implemented by a computing device 114 separate from the measurement apparatus 122. Thus, in certain examples, a measurement apparatus 122 may include communication circuitry 608, instead of or in addition to an analysis module 116. Communication circuitry 608, in the depicted example, is configured to transmit information to a remote data repository 118. The communication circuitry 608 may transmit information via the data network 120, and may include components for data transmission (and possibly reception), such as a network interface controller (NIC) for communicating over an ethernet or Wi-Fi network, a transceiver for communicating over a mobile data network, or the like. Various other or further components for transmitting data may be included in communication circuitry 608 in various other or further examples.

In some examples, the information transmitted by the communication circuitry 608 to the remote data repository 118 may be information based on the plurality of time-dependent measurements performed by the measurement circuitry 606. Information based on the plurality of time-dependent measurements may be the measurements themselves (e.g., raw samples), calculated information based on the measurements (e.g., spectra calculated from the raw data), and/or analysis results (e.g., a characterization) from the analysis module 116. In one or more further examples, an analysis module 116 may be in communication with the remote data repository 118 (e.g., via the data network 120). An analysis module 116 may be configured to characterize one or more parameters of the biochemical interaction based on the information transmitted to the remote data repository 118. For example, instead of the analysis module 116 receiving measurements directly from the measurement circuitry 606, the communication circuitry 608 may transmit measurements (or information about the measurements) to the remote data repository 118, and the analysis module 116 may retrieve the measurements (or information about the measurements) from the remote data repository 118.

In some examples, storing data in a remote data repository 118 may allow information to be aggregated from multiple measurement apparatuses 122 for remote analysis of phenomena that may not be apparent from a single measurement apparatus 122. For example, for epidemiology purposes, a measurement apparatus 122 may determine whether a person is infected with a disease based on a biochemical interaction involving viruses, antibodies, DNA or RNA from a pathogen, or the like, in a sample fluid 110 obtained from the person, which may include a sample of blood, saliva, mucus, cerebrospinal fluid, stool, or the like. Information uploaded to a remote data repository 118 from multiple measurement apparatuses 122 may be used to determine aggregate characteristics, such as how infection rates differ in different geographical regions. In various examples, an analysis module 116 may implement various other or further ways of using aggregate information from multiple measurement apparatuses 122

The measurement apparatus 122, in various examples, may use excitation circuitry 602, measurement circuitry 606, and an analysis module 116 together in various ways with one or more biologically gated transistors 106 to determine or characterize parameters of a biochemical interaction. In some examples, multiple biologically gated transistors 106 may be homogeneously configured (e.g., for redundancy) or heterogeneously configured (e.g., with channel surfaces 428 functionalized in different ways to characterize different aspects of a biochemical interaction).

In one example, the predetermined measurement bandwidth used by the measurement circuitry 606 satisfies a predetermined frequency criterion for measuring at least one or more parameters of the biochemical interaction that will be characterized by the analysis module 116. Motions or interactions of biomolecules or moieties within the sample fluid 110 may occur at a characteristic frequency f1. For example, a CRISPR Cas enzyme may repeatedly attach to and cleave DNA substrate molecules at a characteristic frequency f1. Other motions or interactions of a biomolecule may occur at other characteristic frequencies f2, f3, . . . fn. In some examples, characteristic frequencies for interactions between large biomolecules, or for folding and unfolding of biomolecules may be in a range of 0.1 Hz to 1 kHz. In some examples, characteristic frequencies for internal motions of biomolecules, or for specific binding interaction, may be in a range of 1 kHz to 1 MHz Thus, in certain examples, measurement circuitry 606 that performs time-dependent measurements at a sample rate at least double the characteristic frequency of some motion or interaction may “see” or detect the effects of that motion or interaction on an output signal, and the analysis module 116 may use those measurements to characterize a parameter such as whether, or to what extent, the motion or interaction corresponding to that characteristic frequency occurs.

Accordingly, in some examples, a frequency criterion for measuring at least one parameter of a biochemical interaction may be one or more frequencies for which observation is desired (e.g., one or more characteristic frequencies for the interaction), a band of frequencies, or the like. A frequency criterion may be predetermined by a manufacturer or user of a measurement apparatus 122 based on models of biochemical interactions, prior measurements of biochemical interactions, or the like.

A measurement bandwidth that satisfies a frequency criterion for measuring a parameter of an interaction may be a bandwidth sufficient for the time-dependent measurements to reveal information at the target frequency, frequencies, or frequency band, in the output signals. For example, a measurement bandwidth may satisfy a frequency criterion for observations at frequency f1 if the sample rate for the plurality of time-dependent measurements is at least double the frequency f1. Also, if low frequencies are not observed (e.g., if the measurement bandwidth does not start at zero), a measurement bandwidth may satisfy a frequency criterion for observations in a target range from frequency f1 to frequency fn if the sample rate is double the width of the range. Various other or further ways for a measurement bandwidth to satisfy a frequency criterion may be suggested by application of the Nyquist-Shannon theorem and/or other subject matter related to sampling.

In some examples, excitation circuitry 602 may be configured to vary one or more of the programmable biases applied to a biologically gated transistor 106. For example, the excitation circuitry 602 may use bias circuitry 604 to create and vary a source bias, a drain bias, and/or a gate bias (e.g., applied to a liquid gate via a counter electrode 204 or measured via a reference electrode 208). Varying a programmable bias may include changing the programmable bias over time, where the changes may be discontinuous or continuous. For example, the bias circuitry 604 may increase or decrease a programmable bias in steps, or in a continuous sweep. In some examples, varying the programmable bias may include applying a non-constant bias such as a periodic waveform, which may be a simple waveform such as a sine, cosine, square, triangle, or sawtooth, wave, or which may be a more complex waveform A more complex waveform may be, or may be equivalent to, a sum of sine waves at multiple frequencies referred to as frequency components. Additionally, if the bias circuitry 604 is varies a programmable bias by applying a nonconstant bias such as a periodic waveform, the bias circuitry 604 may further vary the bias by changing the amplitude, frequency, or phase of the waveform, or of one or more frequency components of a complex waveform.

In further examples, the measurement circuitry 606 may perform time-dependent measurements while the excitation circuitry 602 varies one or more of the programmable biases for a biologically gated transistor 106. The measurement circuitry 606 and/or the analysis module 116 may correlate time-dependent measurements with the variations applied by the excitation circuitry 602. For example, the measurement apparatus 122 may include a trigger line to synchronize a function generator for the bias circuitry 604 with the measurement circuitry 606. Also, in some examples, the measurement circuitry 606 may perform time-dependent measurements of one or more programmable biases and one or more output signals. For example, an impedance measurement may include measuring a phase difference between a programmable bias and an output signal.

In various examples, measurement circuitry 606 may perform measurements using a predetermined measurement bandwidth while the excitation circuitry 602 varies programmable biases in various ways. For example, in some examples, the bias circuitry 604 may sweep, scan, or otherwise slowly vary one of the programmable biases while keeping other programmable biases constant (e.g., varying a gate bias at the counter electrode 204 while maintaining a constant drain-to-source voltage, varying a drain bias while maintaining a constant gate-to-source voltage, or varying a source bias while maintaining a constant drain-to-gate voltage), and the measurement circuitry 606 may perform measurements using a predetermined measurement bandwidth (which may be a higher-frequency band than the slow or low-frequency bias variations). Such a slow variation in bias may be part of a complex overall waveform that includes variations at different frequencies in combinations including speeds slower than the measurement bandwidth, within the measurement bandwidth, and faster than the measurement bandwidth.

Choosing frequencies for bias variations involves coordination of the bias circuitry 604 and measurement circuitry 606. Selection of which frequencies to include in programmable biases may be based on measured, typical, or expected timescales for equilibration in the sample as well as measured, typical, or expected resonances and frequency dependence of the liquid dielectric. Slow frequency components can be thought of as variations slow enough that elements and effects in the sample such as faradaic current or double layer reorganization can approximately come to equilibrium in between measurement events. Frequencies within the measurement bandwidth can be thought of as targeting resonances or targeting distances from the surface. Frequencies higher than the measurement bandwidth can be thought of as driving potentials seeking to trigger an interaction or non-linear effect that is then measured at a lower frequency.

In some examples, the bias circuitry 604 may vary more than one of the programmable biases while the measurement circuitry 606 performs measurements. In some cases, it will be desirable to vary one particular voltage over another. For example, applying a varying or high-frequency bias voltage to the liquid gate via the counter electrode will probe the entire region in between the counter electrode and the graphene channel. Appropriate changes in frequency range and analysis, may be used to probe changes in the conductivity of the bulk solution, biochemical or chemical changes in regions far from the surface layer, biochemical or chemical changes within the Donnan layer or double layer. This might be desirable when looking for enzymatic changes in solution, small molecule and cell signaling interactions, metabolic signals, changes in salt or pH.

By contrast, applying a varying or high-frequency bias to the source and/or drain electrode will probe the region closest to the graphene channel. This will be primarily useful for evaluating surface effects, surface chemistry, blocking layers, and other attached biomolecules. For example, attaching an enzyme to the surface and then using a varying or high-frequency bias on the source and/or drain may allow sensitive detection of motions of the enzyme on the surface. Similarly, it may allow evaluation of whether chemical modifications have been made to the surface chemistry, such as binding of a target nucleic acid to the surface chemistry. Controlling or triggering chemistry on the surface can be done with application of either voltage to the liquid gate or voltage to source and/or drain. Applying varying or high-frequency biases to both the liquid gate and source and/or drain can be used to expand measurement opportunities. For example, applying a varying or high-frequency bias the liquid gate via the counter electrode may be used to drive and reverse a large-scale protein motion such as a binding interaction, while a varying or high-frequency bias applied to the source and/or drain may be tuned for detecting/measuring a resonance that only occurs during binding. In this way, multiple repeated measurements can be made rapidly, increasing overall sensitivity.

Also, in some examples, the bias circuitry 604 may modulate one or more of the programmable biases at one or more excitation frequencies while the measurement circuitry 606 performs measurements. For example, to measure a resonance of a biochemical interaction, the bias circuitry 604 may modulate one or more of the programmable biases at a resonant or characteristic frequency for the interaction, and the measurement circuitry 606 performs measurements using a measurement bandwidth that includes the resonant or characteristic frequency.

In some examples, as described above, a measurement distance 502 may be based on or correspond to a frequency in the measurement bandwidth. For example, low-frequency or electrostatic aspects of a biochemical interaction may be screened by a Donnan equilibrium region or an ionic double layer, and thus may affect the output signals when they occur within the electrostatic screening distance 504, but not when they occur further into the bulk of the sample fluid 110. However, higher-frequency aspects of a biochemical interaction may affect the output signals at a measurement distance 502 greater than the electrostatic screening distance 504. Thus, in some examples, measurement circuitry 606 may obtain information corresponding to the biochemical interaction occurring at a desired measurement distance 502 greater than an electrostatic screening distance 504 by using a measurement bandwidth that corresponds to the desired measurement distance 502, even if the programmable biases from the bias circuitry 604 are low-frequency or non-periodic (e.g., constant, or slowly swept).

Additionally, excitation circuitry 602 may vary or modulate one or more of the programmable biases at an excitation frequency. Resonances or characteristic frequencies for the biochemical interaction may affect the time-dependent measurements of output signals more significantly if those resonances are excited by a programmable bias or other excitation condition modulated at a resonant or characteristic frequency. Thus, in some examples, an excitation frequency may be a frequency within the measurement bandwidth, and a measurement distance 502 may correspond to the excitation frequency. Modulating a programmable bias at an excitation frequency may include modulating a programmable bias amplitude by a wave (e.g., a sine, cosine, square or other waveform) at the excitation frequency, or may include varying a programmable bias according to a complex waveform with a frequency component at the excitation frequency. For example, a programmable bias modulated at an excitation frequency f1 may be a wave with a frequency f1. Similarly, a programmable bias modulated at multiple excitation frequencies f1 through fn may be a sum of waves with frequencies f1 through fn. Alternatively, a programmable bias modulated at multiple excitation frequencies may be a sequence of waves with frequencies f1 through fn, applied to a biologically gated transistor 106 sequentially rather than simultaneously, or in some combination of sequential and simultaneous approaches.

Various excitation frequencies may facilitate characterization of various parameters of a biochemical interaction. A cutoff frequency for screening by an ionic double layer may be in a range from approximately 1 to 50 MHz, depending on the content of the sample fluid 110. At excitation frequencies below the cutoff frequency, the effects to be seen may include resonances that could provide a “fingerprint” of the biochemical interaction. For example, the resonant frequency of the oscillation of a biomolecular complex linked to the channel 210 under the applied field will be sensitive to the mass of the complex, and so monitoring this frequency in the measurement bandwidth allows for interrogation of the state of the complex by the analysis module 116.

At excitation frequencies approaching the cutoff frequency for screening by an ionic double layer, the Debye length (or thickness) of the double layer will be affected by the excitation frequency, as the ions begin to lag behind the field. By scanning the applied frequency from low to high frequencies, the measurement distance 502 will increase, providing information about biochemistry occurring at higher distances from the channel surface 428. The signals detected for the different frequencies could be compared to models of the biochemistry by the analysis module 116 to gain information about the interactions occurring.

At excitation frequencies well above the cutoff frequency for screening by an ionic double layer, a biologically gated transistor 106 may be much more sensitive to the response of biomolecules or moieties to an applied field. The resultant signals will not be screened by the double layer, and the dipole resonances of a biomolecular complex may be observed as they modulate the output signals of the biologically gated transistor 106.

In some examples, where the excitation circuitry 602 modulates at least one of the programmable biases at multiple excitation frequencies, the analysis module 116 may characterize changes in the biochemical interaction corresponding to one or more changes between excitation frequencies. For example, in one example, a channel surface 428 may be functionalized with capture or linker molecules to bind to another moiety, such as antibodies that bind to an antigen. However, the antigen may be part of a large particle (such as a pathogen that causes an infectious disease), or may be part of a smaller particle. (such as a fragment of a pathogen). The linker molecule and the linked particle may have a resonance similar to a mass (the linked particle) at the end of a spring (the linker molecule), so analysis of how the output signals change in response to changes in the excitation frequencies, or how the output signals respond to different excitation frequencies may allow the analysis module 116 to distinguish between interactions involving larger captured particles and interactions involving smaller captured particles.

In one or more further examples, where the excitation circuitry 602 modulates at least one of the programmable biases at multiple excitation frequencies, the analysis module 116 is configured to characterize one or more parameters of the biochemical interaction at multiple measurement distances 502 from the surface 428 of the channel 210, where the multiple measurement distances 502 correspond to the multiple excitation frequencies. Because each excitation frequency may affect the interaction at a different distance from the channel surface 428, applying multiple excitation frequencies may allow the analysis module 116 to characterize parameters for the biochemical interaction for different “slices” through the sample fluid 110 at different measurement distances 502.

For example, in one or more examples, a channel surface 428 may be functionalized with linker molecules and antibodies to capture exosomes in the sample fluid 110. Exosomes may be extracellular vesicles bound by a membrane, with a diameter of about 30-150 nm. Excitation circuitry 602 may modulate at least one of the programmable biases at multiple frequencies so that lower frequencies allow the analysis module 116 to characterize parameters of a biochemical interaction relating to the bound exosomes, at a measurement distance 502 close to the channel surface 428, while higher frequencies allow the analysis module 116 to characterize parameters of a biochemical interaction relating to what occurs in the bulk sample fluid 110 at a measurement distance 502 further away from the channel surface 428. Thus, for example, the analysis module 116 may distinguish interactions of moieties bound to the surface of exosome membranes from similar interactions of the same moieties moving freely in the bulk sample fluid 110 based on different excitation frequencies. In some examples, the excitation circuitry 602 may be used to electrokinetically manipulate exosomes or other biomolecules with the sample fluid 110.

In some examples, the excitation circuitry 602 may modulate one or more of the programmable biases at two different excitation frequencies, and the measurement bandwidth may include a heterodyne frequency based on the excitation frequencies. A heterodyne frequency based on two excitation frequencies may be a sum or difference of the two frequencies. For example, the excitation circuitry 602 may modulate one or more of the programmable biases using a first excitation frequency and a second excitation frequency different from the first excitation frequency. The first and second excitation frequencies may be applied to different terminals of a biologically gated transistor 106, or simultaneously to a single terminal. A heterodyne frequency, such as a sum or difference of the first and second excitation frequencies, may be within the measurement bandwidth. For example, the excitation frequencies used to modulate the programmable biases may be outside the measurement bandwidth, but, due to the nonlinear dielectric properties of an attached protein, the frequency of an output signal for a biologically gated transistor 106 may be within the measurement bandwidth. This allows the measurement circuitry 606 to filter out the entirety of the applied biases, lowering the noise and increasing the sensitivity or selectivity of the measurement.

The heterodyne frequency at a sum or difference of the excitation frequencies may result from modulation of the programmable bias(es) by excitation circuitry 602. For example, the excitation circuitry 602 may modulate programmable biases including a source bias, a drain bias, a gate bias applied to the liquid gate via a counter electrode 204, or a combination of source and gate or source and drain biases at two different excitation frequencies, in which one or both of the excitation frequencies is above the cutoff frequency of the chip-based biosensor 104. The response at the heterodyne frequencies may then be measured by measurement circuitry 606.

In some examples, excitation frequencies may be above or below a cutoff frequency for the chip-based biosensor 104 and/or the measurement circuitry 606 or other components of a measurement apparatus 122, and the measurement bandwidth may include a heterodyne frequency. For example, small changes to protein conformation may occur in a nanosecond timescale. Similarly, the relaxation frequency for an ionic double layer may be in a range from 1-100 MHz, depending on the ionic strength of the solution. However, in some examples, a biologically gated transistor 106 may have a cutoff frequency of about 2 MHz Measurements to detect, measure, or “see” events above the 2 MHz cutoff frequency (e.g., events outside the ionic double layer, or events at a nanosecond timescale) may use at least one excitation frequency above the cutoff frequency, and may measure at heterodyne frequencies. Thus, in some examples, using excitation circuitry 602 to apply multi-frequency excitation biases may allow the measurement circuitry 606 to perform measurements and “see” or detect interactions at heterodyne frequencies significantly higher or lower than the excitation frequencies.

In various examples, the excitation circuitry 602 may modulate a programmable bias (or multiple programmable biases) at an excitation frequency, and the measurement bandwidth for the measurement circuitry 606 may include one or more higher harmonics of the excitation frequency (e.g., second harmonics, third harmonics, or the higher, where the first harmonic is the fundamental frequency), when the transfer curve (current versus gate voltage) is nonlinear. For example, a particular excitation frequency might drive enzymatic activity for a certain enzyme. The enzyme performing that activity will make multiple smaller actions, such as binding, chemical modification, and release, to complete a complete enzymatic cycle. Each of these steps will necessarily be performed faster than the drive cycle and will have a higher characteristic frequency. To evaluate only one of the sub-steps of an enzyme activity, a frequency within the measurement bandwidth may be higher than an excitation frequency. In another example, the Dirac voltage for a biologically gated transistor 106 may shift when a particular binding event happens, such as an antigen binding to an antibody immobilized to the channel 210. A varying or high-frequency gate bias may be applied to the liquid gate via the counter electrode 204, with the gate bias centered at a DC offset that matches the shifted Dirac voltage associated with the binding event. After the binding event, the frequency of the applied gate bias is doubled in the current through the channel because of the high nonlinearity in the current versus gate voltage response of the transistor, and can be sensitively measured as a higher harmonic which is not present prior to binding. Similarly, in some examples, the measurement bandwidth for the measurement circuitry 606 may include one or more higher harmonics of a characteristic or resonant frequency for a biochemical interaction, whether or not the excitation circuitry 602 specifically uses that frequency to modulate a programmable bias. Measurement at higher harmonics of an excitation or resonance frequency may provide additional information for characterizing the interaction.

In some examples, certain aspects of a biochemical interaction may be temperature sensitive. Thus, the excitation circuitry 602 may use temperature control circuitry 414 to apply a temperature change to the sample fluid 110. For example, a particular Cas enzyme may work optimally at a particular temperature. In this case, moving the temperature into that optimum range maximizes the sensing signal, while moving the temperature out of that range increases selectivity by verifying that a presumed positive measurement of Cas activity is reduced at a sub-optimal temperature. The measurement circuitry 606 may perform time-dependent measurements before and after the temperature change, and the analysis module 116 may characterize a change in the biochemical interaction corresponding to the temperature change.

In various examples, excitation circuitry 602 and measurement circuitry 606 may perform a control measurement in parallel with a measurement using a first biologically gated transistor 106. For example, a second biologically gated transistor 106 may be provided in a chip-based biosensor 104, with a non-reactive biomolecule blocking layer or a control fluid such as water instead of the sample fluid 110. The excitation circuitry 602 and the measurement circuitry 606 may apply excitations and perform measurements for both transistors 106 in parallel, and the control measurements from the second biologically gated transistor 106 may be subtracted from the measurements from the first biologically gated transistor 106 prior to analysis by the analysis module 116.

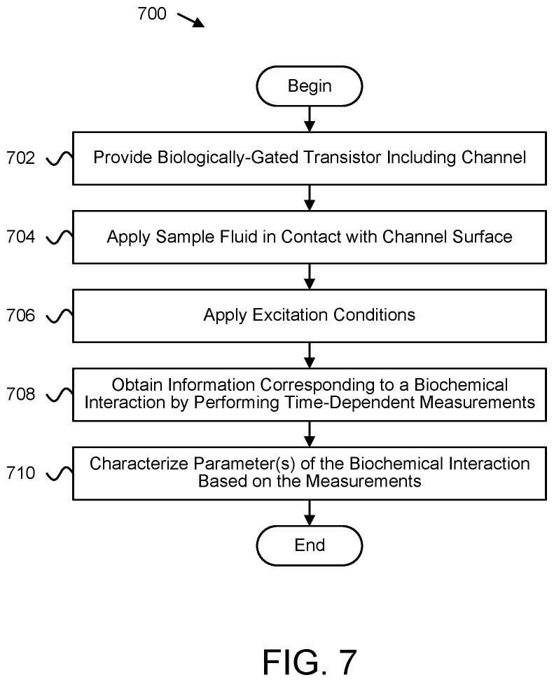

FIG. 7 is a schematic flow chart diagram illustrating a method 700 for excitation and measurement of biochemical interactions, in accordance with one or more examples of the present disclosure. The method 700 begins with providing 702 a biologically gated transistor 106 including a channel 210. A sample fluid 110 is applied 704 to the biologically gated transistor 106 in contact with a surface 428 of the channel 210. Excitation circuitry 602 applies 706 one or more excitation conditions to the biologically gated transistor 106, so that one or more output signals of the biologically gated transistor 106 are affected by a biochemical interaction within the sample fluid 110. In some examples, the excitation conditions include a plurality of programmable biases including a gate bias applied by bias circuitry 604 to a liquid gate of the biologically gated transistor 106 (e.g., via a counter electrode 204) and a drain bias applied to a drain 202 of the biologically gated transistor 106. In some examples, applying 706 the excitation conditions may include the excitation circuitry 602 modulating one of the programmable biases at multiple excitation frequencies.

Measurement circuitry 606 obtains 708 information corresponding to the biochemical interaction by performing a plurality of time-dependent measurements of at least one of the one or more output signals affected by the biochemical interaction, using a predetermined measurement bandwidth corresponding to one or more measurement distances. An analysis module 116 characterizes 710 one or more parameters of the biochemical interaction based on the plurality of time-dependent measurements, and the method 700 ends. In some embodiments, characterizing 710 one or more parameters of the biochemical interaction may include the analysis module 116 characterizing one or more changes in the biochemical interaction, corresponding to one or more changes between multiple excitation frequencies.

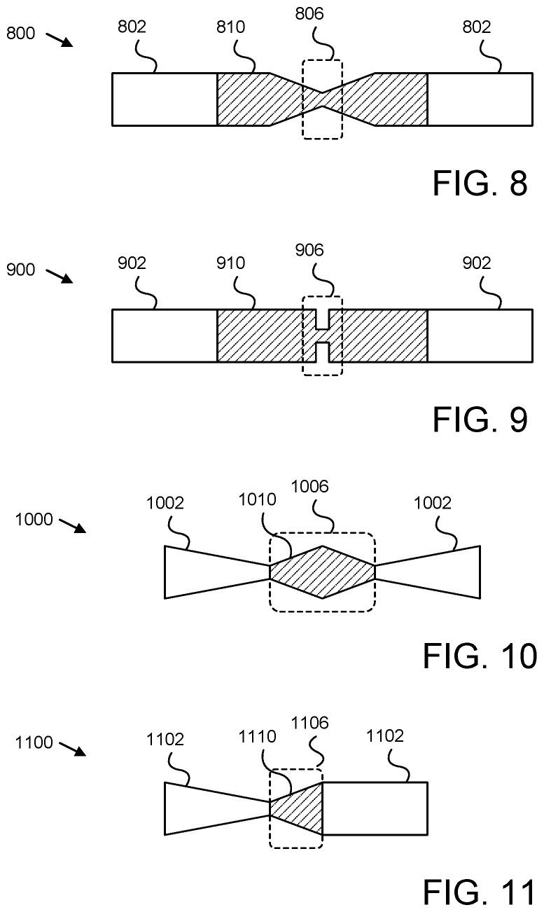

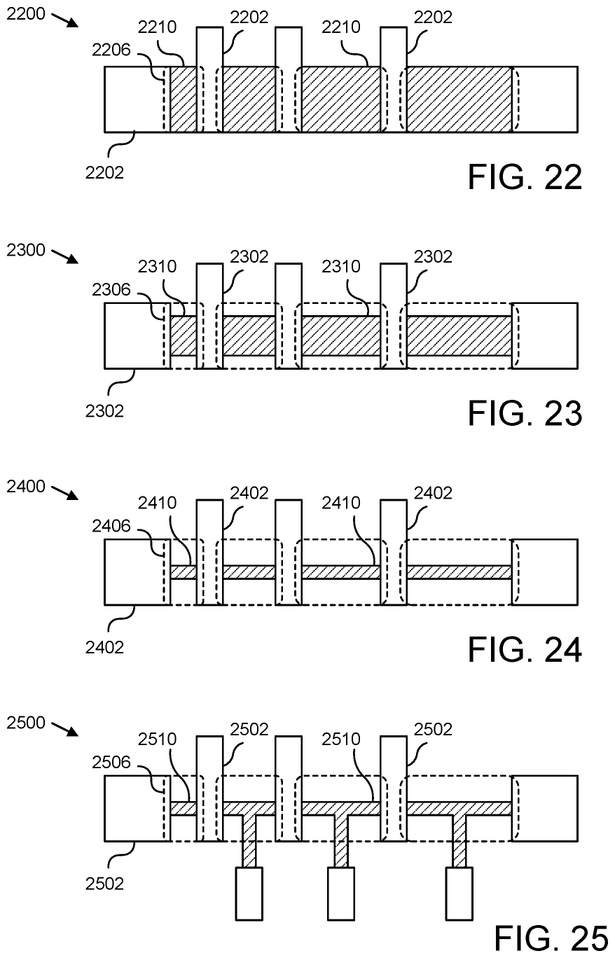

FIGS. 8-30 depict various examples of one or more liquid-gated graphene field effect transistors (“gFETs”). The gFETs depicted in FIGS. 8-30 may be substantially similar to the biologically gated transistors 106, 106a, 106b, 106c described above with reference to FIGS. 1-4, apart from differences with are described below.

Referring to FIG. 2, a liquid-gated transistor 106 includes a channel 210 coupling a source contact 212 to a drain contact 202, so that one or more output signals for the transistor are affected by excitation conditions and by one or more ions, molecules, or moieties within a sample fluid in contact with, or within detection range or the channel. Similarly, in the liquid-gated gFETs described with reference to FIGS. 8-30 a channel conducts electrical current between contacts, and output signals may be affected by events or interactions in a fluid in contact with the channel. Certain components of liquid-gated graphene field effect transistors are omitted from FIGS. 8-30 for convenience in depicting variations between other components, but may nevertheless be present in actual transistors. For example, FIGS. 8-15 and 19-30 do not depict reference electrodes or counter electrodes, but actual transistors including the components depicted in these figures may include reference electrodes and counter electrodes.

Additionally, in FIGS. 8-30, contacts for conducting electrical current into or out of a channel (such as contacts 802 of FIG. 8 are not labeled as source or drain contacts, as current may flow in either direction depending on a bias between the contacts, and with majority charge carriers as electrons or holes depending on applied gate voltage (e.g., via a counter electrode) and/or other conditions in the applied sample fluid. Nevertheless, contacts described with reference to FIGS. 8-30 may be substantially similar to the drain 202 and source 212 contacts described above for other transistors.

Referring to FIG. 8, a gFET 800 includes at least two contacts 802 coupled by a graphene channel 810. A passivation layer is deposited over portions of the contacts 802 and/or the channel 810, and a window 806 (indicated by a dashed line) is patterned in the passivation layer to expose at least a portion of the channel 810. In some examples, a passivation layer may expose small portions of the contacts 802.

In subsequent figures, like numbers refer to like elements unless otherwise clear from context. Thus, in FIG. 9, one example of a gFET 900 includes contacts 902 coupled by a channel 910, with a window 906 in a passivation layer exposing at least a portion of the channel 910. Subsequent FIGS. 10-30 similarly depict transistors 1000, 1100, 1200, 1300, 1400, 1500, 1600, 1700, 1800, 1900, 2000, 2100, 2200, 2300, 2400, 2500, 2600, 2700, 2800, 2900, 3000; which respectively include two or more contacts 1002, 1102, 1202, 1302, 1402, 1502, 1602, 1702, 1802, 1902, 2002, 2102, 2202, 2302, 2402, 2502, 2602, 2702, 2802, 2902, 3002; coupled by channels 1010, 1110, 1210, 1310, 1410, 1510, 1610, 1710, 1810, 1910, 2010, 2110, 2210, 2310, 2410, 2510, 2610, 2710, 2810, 2910, 3010; and where a window 1006, 1106, 1206, 1306, 1406, 1506, 1606, 1706, 1806, 1906, 2006, 2106, 2206, 2306, 2406, 2506, 2606, 2706, 2806, 2906, 3006 in a passivation layer exposes at least a portion of the channel surface.

In some examples, a gFET channel may be rectangular, as depicted in FIG. 2. However, various transistor shapes and designs described below with reference to FIGS. 8-30 may affect different types of measurements (as described herein for biologically gated transistors 106) in various ways.

FIG. 8 depicts a gFET 800 with a constriction-based design, where the graphene is patterned so that the channel width 810 is gradually reduced to come to a minimum width at one point along the channel. This channel width could be less than 100 nanometers, which would start creating a constriction driven bandgap in the graphene and steepening the slope of the gate transfer curve (e.g., on an I-V graph of current between contacts 802 versus a gate voltage applied via a counter electrode). Such a constriction-based device might have some frequency dependence to the bandgap, and may also cause any biology or other sensing target located at the constriction to dominate a sensing measurement, simplifying the analysis of frequency-based measurements by limiting the source of chemical interactions to a small number of sites, potentially to a single site.

In a less extreme example of the constriction-based design, the constriction would be used not to create a bandgap, but just to reduce the active gate area of the transistor 800 (e.g., gFET) in a manner that is easy to fabricate. For example, optical lithography will be limited to a resolution of 0.2-1.0 micrometers, depending on the tool used. In this case, a horizontal constriction would be patterned down close to the resolution of the tool, and then this would be interfaced with a vertical window 806 also patterned at the resolution in the gate passivation to give a total liquid gate overlap of roughly the square of the optical lithography resolution. This small liquid gate region will have a reduced capacitance, thereby increasing the speed of the device, and the graphene exposed area available for functionalization will be small, which could be a route towards single molecule detection.

FIG. 9 depicts a gFET 800 with a small channel design, where the channel 910 includes a small generally rectangular region inserted in between two larger graphene regions. This design has many of the same properties as the constriction-based design, but may be easier to fabricate.

For some fabrication methods, the size of the constriction or small channel may be limited by lithography resolution. However, certain etching techniques may be used to form smaller graphene regions for a constriction or small channel. For example, a “hardmask” layer of gold or other suitable metal may be deposited over a graphene channel 910 to protect the channel from contamination or damage during subsequent processes such as patterning of a passivation layer to form a window 906. The gold may subsequently be etched to expose the channel 810. However, if the hardmask (e.g., gold) were made very thin, e.g., around 10 nanometers, the undercut etch rate of the metal would be slow and the graphene area could be reduced controllably below optical resolution by using a controlled wet etch of the metal. Alternatively, different types of protective layers, such as a layer of aluminum oxide on top of an even thinner layer of metal (e.g., gold), could be etched sequentially.

FIGS. 10 and 11 depict gFETs 1000, 1100 with contact limited designs. In these cases, instead of the minimum channel width occurring in the middle of the channel 1010, 1100, it would occur at one or both contacts 1002, 1102 with the source and drain metal leads. FIG. 10 depicts a symmetric design with minimum channel width occurring at both contacts 1002, while FIG. 11 depicts an asymmetric design with minimum channel width occurring at one contact 1102. In these cases, the contact location would be a dominating point of resistance.

Referring to FIG. 10, in a symmetric contact limited channel 1010, the self-gating of the source and drain contacts 1002 would lead to gradual and reversible non-linear behavior in resistance versus source-drain voltage. The gate transfer curve could also be skewed to n-type or p-type by applying larger voltages relative to the gate to either the source or the drain contacts 1002. This may allow for precise tuning of non-linear effects to enhance desired frequency mixing or to reduce undesired frequency mixing. In addition, the design of FIG. 10 allows for changing the ratio of the quantum capacitance of the transistor to the geometric capacitance of the transistor. The quantum capacitance will be limited by the constricted source and drain, while the geometric capacitance will be enhanced by a larger surface area. Changing this ratio will allow for engineering the relative contribution of the sample liquid composition to the electrical properties of the overall system. FIGS. 8 and 9 show examples of how to enhance the contribution of the quantum capacitance relative to the geometric capacitance.

Referring to FIG. 11, an asymmetric contact limited channel 1110 would only have a channel constriction at one contact 1102. This would lead to a device that is easier to control as only one voltage relative to gate would need to be properly controlled to obtain a desired nonlinear response. Nonlinear output curves might also be achieved or enhanced by using two different metals with very different work functions, such as titanium and nickel to form contacts 1102 on opposite sides of the channel 1110.

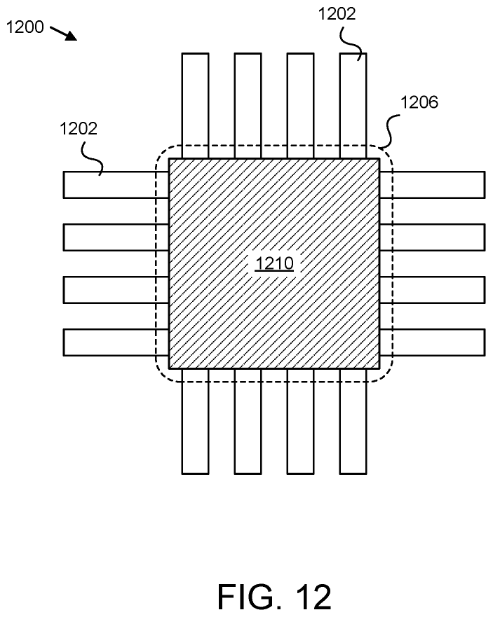

FIG. 12 depicts a gFET 1200 in a tomographic transistor design, where the channel 1210 is a is a relatively large sheet of graphene with multiple contacts 1202. A combination of source voltages and current measurements with different contacts 1202 would allow a mapping of resistance continuously across the graphene sheet in two dimensions. The resolution of the mapping is set by the interelectrode spacing on the edges. This design may facilitate multiplexing, with large multiplex arrays only limited by how closely distinct binding moieties could be attached to the multiplexed portions of the graphene surface. The fabrication of a multiplexed tomographic transistor design is simpler that various multiplexed transistor designs. The tomographic transistor design of the gFET 1200 has enhanced usefulness for on-chip spatial sorting and/or separation of the analytes. Another advantage of this design is a significant reduction in surface buffering effects of non-graphene materials exposed by the window 1206, due to the fact that a large proportion of the surface area within the window 1206 is graphene.

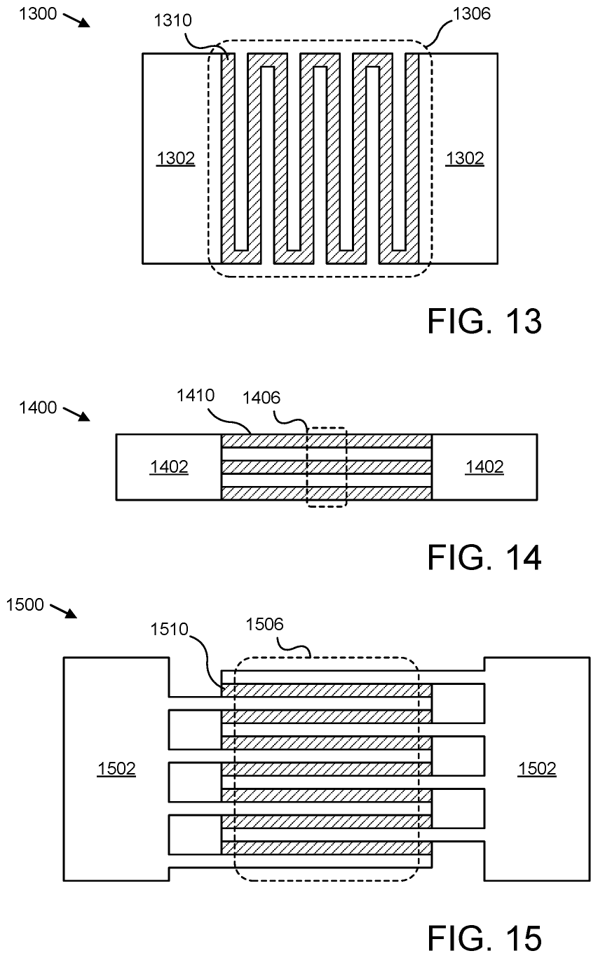

FIG. 13 depicts a gFET 1300 with a serpentine-shaped channel 1310. The serpentine channel 1310 provides a long graphene channel with a large sensing area in a compact shape suitable for pixel multiplexing. This design may facilitate sensing in samples with high dilutions, although it may have a low transconductance. The channel 1310 may also have a larger edge to surface plane ratio than non-serpentine channels, which may result in increased sensitivity in applications where the more chemically reactive edges of a channel 1310 are used for functionalization and sensing. Drawbacks of this design include high overall resistance, low transconductance, and sensitivity to fabrication problems. In terms of impedance, this design may have high resistance, high inductance, and high gate capacitance.

FIG. 14 depicts a gFET 1400 in which the channel 1410 comprises parallel strips of graphene. Compared to the serpentine channel 1310 depicted in FIG. 13, a parallel strip design for a channel 1410 may similarly have a high edge to surface plane ratio for the graphene, but with a higher transconductance.

FIG. 15 depicts a gFET 1500 where the channel 1510 comprises parallel graphene strips disposed between interdigitated contacts 1502. Interdigitated contacts 1502 may provide a large channel width and large sensing area in a compact shape suitable for multiplexing. In this case the transconductance may be very high, and the graphene planar surface area to edge ratio may be large, which may result in an increased signal to noise ratio. The fabrication of this kind of device may be more difficult for small lengths, in which case reducing or minimizing the contact resistance may retain sensitivity of the transistor 1500 to binding events in the channel 1500. This design may have low resistance, low inductance, and high gate capacitance.

Various examples of gFETs described in FIGS. 8-15 may facilitate time-dependent measurement of output signals at certain measurement frequencies, because they allow for design of device impedances. For example, selecting certain sizes and shapes for contacts or the channel of a gFET may allow a manufacturer to customize the total resistance, the contact resistance, the channel resistance, the channel inductance, and/or the channel capacitance (to gate).

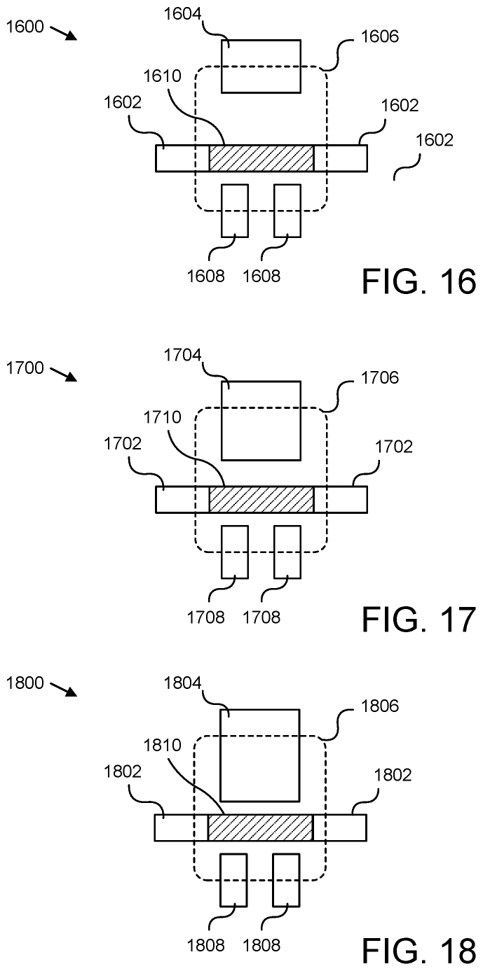

FIGS. 16-30 depict gFETs designs that may be used to test various properties of transistor materials, channel resistivity. Such designs may also be useful in multiplex sensing applications, where providing gFETs with varying properties may facilitate various kinds of measurements.

FIGS. 16-18 depict gFETs 1600, 1700, 1800 including counter electrodes 1604, 1704, 1804 and dual reference electrodes 1608, 1708, 1808, which may be substantially similar to counter electrodes 204 and reference electrodes 208 described above. The distance between the counter electrodes 1604, 1704, 1804 and the channels 1610, 1710, 1810 decreases from gFET 1600, to gFET 1700, to gFET 1800. Providing multiple transistors with different channel-to-counter electrode spacings may facilitate the measurement of the liquid gate resistance. Providing more than one platinum reference electrode 1608, 1708, 1808, may facilitate measuring the stability of individual reference electrodes, or may facilitate mapping the spatial variance of the potential of the applied liquid if the resistivity of the applied liquid is high.

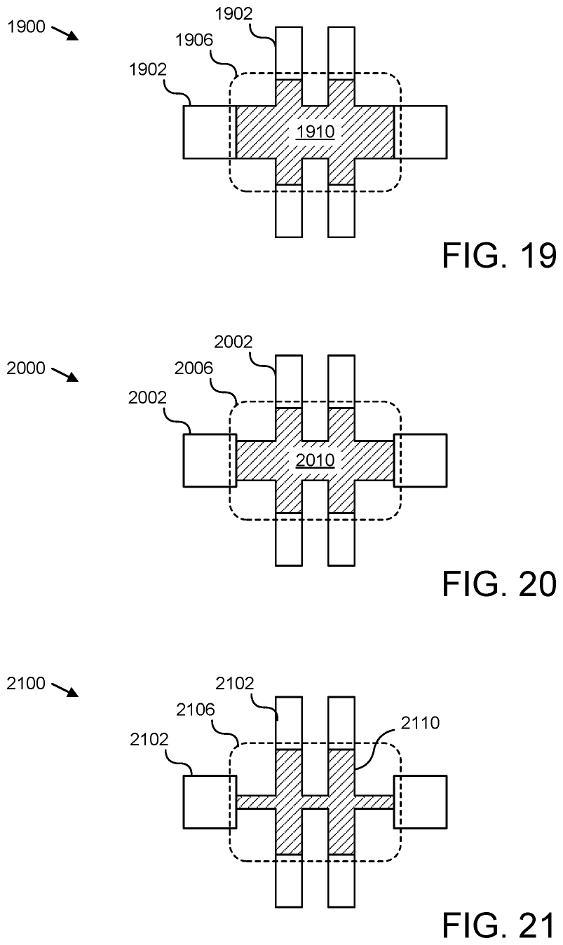

FIGS. 19-25 depict gFETs for measuring the resistivities, including contact resistivity and graphene sheet resistance, as a function of channel width. In FIGS. 19-21, the “Hall bar” geometry of gFETs 1900, 2000, 2100 allows for four-point probe techniques and for Hall resistance measurements under a magnetic field, to determine the density of charge carriers in the graphene channels 1910, 2010, 2110. The width of the graphene channels 1910, 2010, 2110 decreases from gFET 1900, to gFET 2000, to gFET 2100, allowing measurements to be made as a function of channel width.

In FIGS. 22-24, transmission line measurement (TLM) geometry of gFETs 2200, 2300, 2400 includes multiple channels 2210, 2310, 2410 between contacts, with the channel length varying within each transistor, allowing measurements to be made as a function of channel length. As in FIGS. 19-21, channel width varies between transistors 2200, 2300, 2400, allowing measurements to be made as a function of channel width. FIG. 25 depicts a gFET 2500 with a hybrid Hall bar and TLM design.

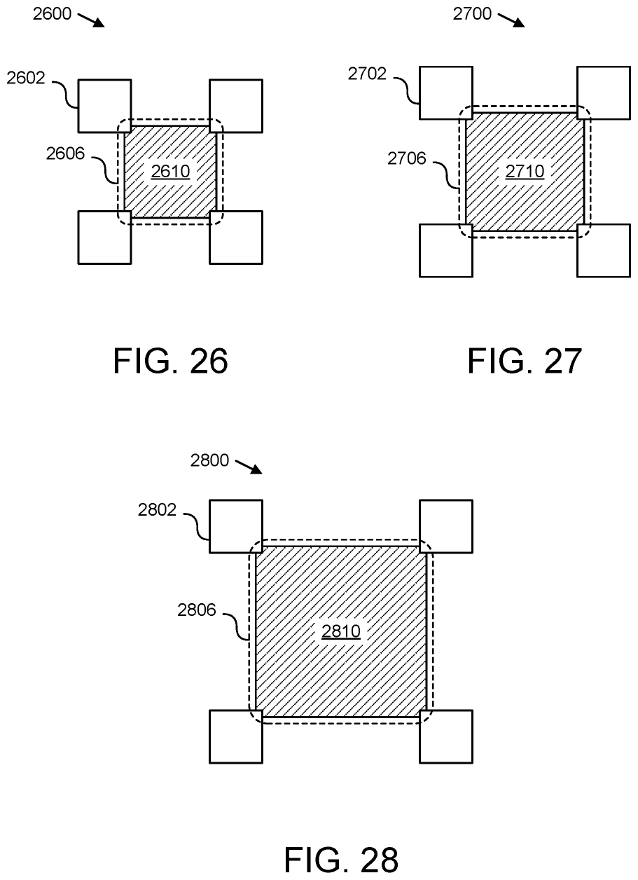

FIGS. 26-28 depict gFETs 2600, 2700, 2800 configured as van der Pauw structures that would allow for the variation of channel area with a constant width to length ratio, to provide a constant resistance. The van der Pauw structures are four-point probe structures, allowing for the measurement of resistivity.

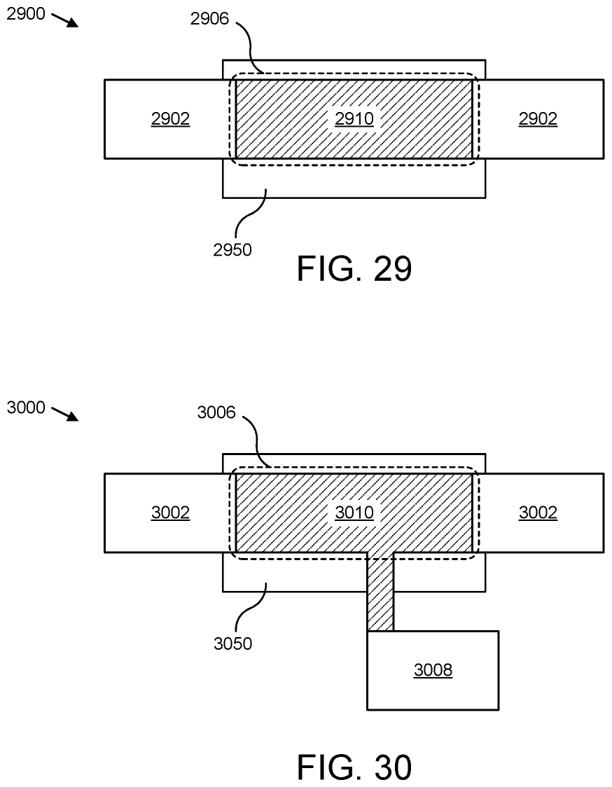

FIGS. 29-30 depict gFETs 2900, 3000 as locally backgated structures. Chemical-mechanical polishing (“CMP”) may be used to fabricate a local back gate 2950, 3050 under the graphene channel 2910, 3010. This would allow for the variation of the channel surface potential and liquid potential somewhat independently. Conversely, local back gates 2950, 3050 may be used to link the channel potential and liquid potential, or gating independently of a reference electrode, which means that the channel could be used as a reference electrode, rather than as a working electrode. In that case, source and drain contacts 2902, 3002 may be capacitively coupled to the chip wiring to allow the graphene channel potential to float at the DC liquid voltage. High frequency excitation and measurement could provide alternating current through the channel, via the capacitive coupling of the source and drain contacts to the chip wiring.

As depicted in FIG. 30, the graphene channel 3010 may be connected to an additional platinum reference electrode 3008 to more closely match the graphene channel potential to the liquid potential. Minimizing the potential difference between the graphene and the liquid may protect the graphene from damage in cases where large potentials are applied to the liquid, such as when electrophoresis is performed. For example, in some example arrays, electrophoretic methods using large potential may be used to functionalize predetermined transistors with different capture agents by moving the different capture agents horizontally and/or vertically to associate to the predetermined transistors.

It may be noted that various combinations of a portion or all of the first through twenty third geometries depicted in FIGS. 8-30 may be utilized as heterogeneous and compatible building blocks that may be used to form distinct single transistors, groups of transistors, multichannel transistors, arrays of transistors, which may be heterogeneously or homogeneously functionalized to accommodate various modes of excitation, measurement frequencies, multiplexing, and the like.

Examples and implementations may be practiced in other specific forms. The described examples are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An apparatus comprising:
excitation circuitry configured to apply one or more excitation conditions to a biologically gated transistor comprising a channel, such that one or more output signals from the biologically gated transistor are affected by the one or more excitation conditions and by a biochemical interaction of moieties within a sample fluid in contact with a surface of the channel;
measurement circuitry configured to obtain information corresponding to the biochemical interaction occurring at one or more measurement distances including at least one measurement distance greater than an electrostatic screening distance from the surface of the channel, by performing a plurality of time-dependent measurements of at least one of the one or more output signals affected by the excitation conditions and the biochemical interaction, using a predetermined measurement bandwidth corresponding to the one or more measurement distances; and
an analysis module configured to characterize one or more parameters of the biochemical interaction based on the plurality of time-dependent measurements.

2. The apparatus of claim 1, wherein the predetermined measurement bandwidth satisfies a predetermined frequency criterion for measuring at least one or more parameters of the biochemical interaction.

3. The apparatus of claim 1, wherein the excitation conditions comprise a plurality of programmable biases including a gate bias applied to a liquid gate of the biologically gated transistor, a drain bias applied to a drain of the biologically gated transistor, and a source bias applied to a source of the biologically gated transistor, and the excitation circuitry is configured to vary one or more of the programmable biases.

4. The apparatus of claim 3, wherein the excitation circuitry is configured to modulate one of the programmable biases at multiple excitation frequencies and the analysis module is configured to characterize one or more parameters of the biochemical interaction at multiple measurement distances from the surface of the channel, the multiple measurement distances corresponding to the multiple excitation frequencies.

5. The apparatus of claim 3, wherein the excitation circuitry is configured to vary one or more of the programmable biases using a first excitation frequency and a second excitation frequency different from the first excitation frequency, and the measurement bandwidth includes at least one heterodyne frequency based on the first excitation frequency and the second excitation frequency.

6. The apparatus of claim 3, wherein the excitation circuitry is configured to modulate one of the programmable biases at an excitation frequency and the measurement bandwidth includes at least one higher harmonic of the excitation frequency.

7. The apparatus of claim 1, wherein the plurality of time-dependent measurements includes measurements of an electrochemical potential of the sample fluid via a reference electrode of the biologically gated transistor.

8. The apparatus of claim 7, wherein the excitation circuitry is configured to adjust the electrochemical potential of the sample fluid by varying a voltage applied to a counter electrode of the biologically gated transistor.

9. The apparatus of claim 1, wherein the excitation conditions comprise a temperature for the sample fluid, and the excitation circuitry comprises temperature control circuitry configured to control the temperature.

10. The apparatus of claim 9, wherein the plurality of time-dependent measurements comprise measurements before and after a temperature change applied by the excitation circuitry, and the analysis module is configured to characterize a change in the biochemical interaction corresponding to the temperature change.

11. The apparatus of claim 1, wherein the analysis module is configured to characterize one or more parameters of the biochemical interaction by determining an observed spectrum based on the plurality of time-dependent measurements and comparing the observed spectrum to one or more model spectra corresponding to one or more models of biochemical interactions.

12. The apparatus of claim 1, further comprising communication circuitry configured to transmit information based on the plurality of time-dependent measurements to a remote data repository.

13. A system comprising:
a biologically gated transistor comprising a channel configured such that one or more output signals of the biologically gated transistor are affected by a biochemical interaction within a sample fluid, in response to application of the sample fluid in contact with a surface of the channel and application of one or more excitation conditions to the biologically gated transistor;
excitation circuitry configured to apply the one or more excitation conditions to the biologically gated transistor;
measurement circuitry configured to obtain information corresponding to the biochemical interaction occurring at one or more measurement distances including at least one measurement distance greater than an electrostatic screening distance from the surface of the channel, by performing a plurality of time-dependent measurements of at least one of the one or more output signals affected by the biochemical interaction, using a predetermined measurement bandwidth corresponding to the one or more measurement distances; and
communication circuitry configured to transmit information based on the plurality of time-dependent measurements to a remote data repository.

14. The system of claim 13, further comprising an analysis module in communication with the remote data repository, wherein the analysis module is configured to characterize one or more parameters of the biochemical interaction based on the information transmitted to the remote data repository.

15. The system of claim 13, wherein the predetermined measurement bandwidth satisfies a predetermined frequency criterion for measuring at least one or more parameters of the biochemical interaction.

16. The system of claim 13, wherein the excitation conditions comprise a plurality of programmable biases including a gate bias applied to a liquid gate of the biologically gated transistor, a drain bias applied to a drain of the biologically gated transistor, and a source bias applied to a source of the biologically gated transistor, and the excitation circuitry is configured to vary one or more of the programmable biases.

17. The system of claim 16, wherein the excitation circuitry is configured to modulate one of the programmable biases at multiple excitation frequencies and an analysis module is configured to characterize one or more parameters of the biochemical interaction at multiple measurement distances from the surface of the channel, the multiple measurement distances corresponding to the multiple excitation frequencies.

18. The system of claim 13, wherein the excitation conditions comprise a temperature for the sample fluid, and the excitation circuitry comprises temperature control circuitry configured to control the temperature.

19. A method comprising:
providing a biologically gated transistor comprising a channel;
applying a sample fluid to the biologically gated transistor in contact with a surface of the channel;
applying one or more excitation conditions to the biologically gated transistor such that one or more output signals of the biologically gated transistor are affected by a biochemical interaction within the sample fluid;
obtaining information corresponding to the biochemical interaction by performing a plurality of time-dependent measurements of at least one of the one or more output signals affected by the biochemical interaction, using a predetermined measurement bandwidth corresponding to one or more measurement distances; and
characterizing one or more parameters of the biochemical interaction based on the plurality of time-dependent measurements.

20. The method of claim 19, wherein:
the excitation conditions comprise a plurality of programmable biases including a gate bias applied to a liquid gate of the biologically gated transistor and a drain bias applied to a drain of the biologically gated transistor;
applying the excitation conditions comprises modulating one of the programmable biases at multiple excitation frequencies; and
characterizing one or more parameters of the biochemical interaction comprises characterizing one or more changes in the biochemical interaction corresponding to one or more changes between excitation frequencies of the multiple excitation frequencies.
